(19) United States
(12) Patent Application Publication
(10) Pub. No.: US 2026/0286030 A1
(43) Pub. Date: Sep. 24, 2026

(54) CRYSTALLIZATION-DRIVEN SELF-STRENGTHENING POLYMERS AND ASSOCIATED COMPOSITIONS AND METHODS

(71) Applicant: THE REGENTS OF THE UNIVERSITY OF COLORADO, a body corporate, Denver, CO (US)

(72) Inventors: Devatha P. NAIR, Denver, CO (US); Dixa GAUTAM, Aurora, CO (US); Gannon KEHE, Lakewood, CO (US); Jeffrey W. STANSBURY, Centennial, CO (US)

(21) Appl. No.: 19/475,460

(22) PCT Filed: Apr. 19, 2024
(86) PCT No.: PCT/US2024/025423

§ 371 Date: Oct. 15, 2025

Related U.S. Application Data
(60) Provisional application No. 63/460,325, filed on Apr. 19, 2023.

(51) Int. Cl. C08F 220/18; C08F 220/06; C08F 220/28; C08F 220/56; C08F 293/00; C08L 33/02; C08L 33/06; C08L 33/08; C08L 33/26; C08L 53/00

(52) U.S. Cl. CPC C08F 220/1818; C08F 220/06; C08F 220/1808; C08F 220/281; C08F 220/56; C08F 293/005; C08L 33/02; C08L 33/066; C08L 33/08; C08L 33/26; C08L 53/00; C08F 2438/03; C08F 2800/10; C08F 2810/20; C08F 2810/50

(57) ABSTRACT

A functionalized crosslinked acrylate copolymer is prepared by partially copolymerizing a composition that includes a first monomer that is monofunctional and has hydrophobic chain, a second monomer that is monofunctional and does not have a hydrophobic chain, and a third monomer that is di-, tri-, or poly functional. Relative to a corresponding copolymer without units derived from the first monomer, the present copolymer provides increased strength as-polymerized, and a further increase in strength over time on exposure to polar solvents, including water.

### CROSS REFERENCE TO RELATED APPLICATIONS

This application claims the benefit of U.S. Application No. 63/460,325, filed on Apr. 19, 2023, which is incorporated herein by reference in its entirety.

### BACKGROUND OF THE INVENTION

Composite restorations have an average lifetime of 6 to 7.2 years and the primary reason for replacement, over the past thirty-five years, has continued to be the downstream effects that arise from a weak adhesive interface. M. Cadenaro et al., “The role of polymerization in adhesive dentistry,” Dental Materials 2019, volume 35, pages e1-e22. Adhesive resins play a pivotal role in anchoring and effectively sealing aesthetically pleasing composite restorations. The composite is too viscous and too hydrophobic to effectively wet and directly bond to the tooth surface, so the function of the low viscosity, more hydrophilic dental adhesive sandwiched between the tooth and the composite layer is to seal the tooth from elements in the surrounding dynamic oral environment. P. Spencer et al., “Threats to adhesive/dentin interfacial integrity and next generation bio-enabled multifunctional adhesives,” Journal of Biomedical Materials Research—Part B Applied Biomaterials, 2019, volume 107, pages 2673-2683; M. Cadenaro et al., “The role of polymerization in adhesive dentistry,” Dental Materials, 2019, volume 35, pages e1-e22. However, the adhesive is the weak link in the restoration and the failure of the restoration at the adhesive interface is responsible for dentin hypersensitivity, recurrent caries, marginal staining, and microleakages, all factors that contribute to the growing clinical burden associated with failed restorations and patient discomfort/dissatisfaction. P. Spencer et al., “Threats to adhesive/dentin interfacial integrity and next generation bio-enabled multifunctional adhesives,” Journal of Biomedical Materials Research—Part B Applied Biomaterials, 2019, volume 107, pages 2673-2683; N. Fraihat, S. Madae'En, Z. Bencze, A. Herczeg, and O. Varga, “Clinical effectiveness and cost-effectiveness of oral-health promotion in dental caries prevention among children: Systematic review and meta-analysis,” International Journal of Environmental Research and Public Health, 2019, volume 16, no. 15, page 2668. The loss of retention of the restoration and secondary caries at the adhesive margins leading to the replacement of existing restorations accounts for 50%-70% of all restorations. H. Mitwalli et al., “Emerging Contact-Killing Antibacterial Strategies for Developing Anti-Biofilm Dental Polymeric Restorative Materials,” Bioengineering, 2020, volume 7, page 83.

The onslaught of challenges to the integrity of the adhesive network begins during the placement of the adhesive layer on the etched and exposed dentin. Low viscosity and ethanol miscibility are required to ensure optimal infiltration of both the interfibrillar spaces of the collagen network and dentinal tubules within the hybrid layer to effectively bond the adhesive layer to the tooth. P. Spencer et al., “Threats to adhesive/dentin interfacial integrity and next generation bio-enabled multifunctional adhesives,” Journal of Biomedical Materials Research—Part B Applied Biomaterials, 2019, volume 107, pages 2673-2683. Toward this end, hydrophilic monomers such as HEMA (2-hydroxyethyl methacrylate) are incorporated within adhesives and together with solvents, dehydrate the dentin by displacing fluids from the dentinal tubules and demineralized collagen networks. K. L. Van Landuyt et al., “The role of HEMA in one-step self-etch adhesives,” Dental Materials, 2008, volume 24, pages 1412-1419. HEMA has low reactivity and forms hydrolytically unstable homopolymers, and therefore crosslinking HEMA with hydrophobic methacrylates that show sufficient reactivity in free-radical photopolymerization, such as bisphenol A glycerolate dimethacrylate (BisGMA) and triethyleneglycol dimethacrylate (TEGDMA), is practiced. K. L. Van Landuyt et al., “The role of HEMA in one-step self-etch adhesives,” Dental Materials, 2008, volume 24, pages 1412-1419; X. Guo, Y. Wang, P. Spencer, Q. Ye, and X. Yao, “Effects of water content and initiator composition on photopolymerization of a model BisGMA/HEMA resin,” Dental Materials, 2008, volume 24, pages 824-831. The resulting polymer network is primarily a heterogeneous one with little to no long-range order with a propensity towards water-induced phase separation, both during photopolymerization and during its lifetime in the aqueous oral environment. X. Guo, Y. Wang, P. Spencer, Q. Ye, and X. Yao, “Effects of water content and initiator composition on photopolymerization of a model BisGMA/HEMA resin,” Dental Materials, 2008, volume 24, pages 824-831. Efforts to increase the strength of the adhesive by increasing the crosslinking density of the network is negated by the early vitrification that occurs during photopolymerization, at which point there is a relatively low degree of methacrylic double-bond conversion. L. Song et al., “Multifunctional monomer acts as co-initiator and crosslinker to provide autonomous strengthening with enhanced hydrolytic stability in dental adhesives,” Dental Materials, 2020, volume 36, pages 284-295. While the low-double bond conversion aids in adhering the composite to the adhesive layer, ultimately, the adhesive layer is akin to a permeable membrane, further weakened by the infiltration of water over time. P. Spencer et al., “Threats to adhesive/dentin interfacial integrity and next generation bio-enabled multifunctional adhesives,” Journal of Biomedical Materials Research—Part B Applied Biomaterials, 2019, volume 107, pages 2673-2683; L. Song et al., “Multifunctional monomer acts as co-initiator and crosslinker to provide autonomous strengthening with enhanced hydrolytic stability in dental adhesives,” Dental Materials, 2020, volume 36, pages 284-295.

Suboptimal photopolymerization of the adhesive layer, high permeability of the bonded interface to water, and attack from cariogenic bacteria are all factors that currently reduce the longevity of the bonded interface and by extension, the restoration. L. Breschi et al., “Dental adhesion review: Aging and stability of the bonded interface,” Dental Materials, 2008, volume 24, pages 90-101. In addition to the economic costs associated with placing/replacing over 800 million composite restorations worldwide, replacement and removal of restorations come at a loss of sound tooth structure that then leads to pulpal injury and further weakening of the tooth. M. Cadenaro et al., “The role of polymerization in adhesive dentistry,” Dental Materials, 2019, volume 35, pages e1-e22; A. Tsujimoto et al., “Wear of resin composites: Current insights into underlying mechanisms, evaluation methods and influential factors,” Japanese Dental Science Review, 2018, volume 54, pages 76-87. The repeated loss of tooth structure and costly dental procedures are not in the best interest of the patient. There remains a need for adhesive formulations that enable more durable and longer-lasting restorations.

### BRIEF SUMMARY OF EMBODIMENTS OF THE INVENTION

One embodiment is a functionalized crosslinked acrylate copolymer comprising: 15 to 63 mole percent of first units derived from a first monomer selected from the group consisting of C₁₀-C₂₄-alkyl (meth)acrylates, C₁₀-C₂₄-alkenyl (meth)acrylates, C₁₀-C₂₄-alkyl (meth)acrylamides, C₁₀-C₂₄-alkenyl (meth)acrylamides, and combinations thereof; 35 to 83 mole percent of second units derived from a second monomer selected from the group consisting of (meth)acrylic acid, C₁-C₈-alkyl (meth)acrylates, C₁-C₈-alkyl (meth)acrylamides, 2-carboxyethyl (meth)acrylate, C₂-C₈-hydroxyalkyl (meth)acrylates, C₂-C₈-hydroxyalkyl (meth)acrylamides, ethylene glycol methyl ether (meth)acrylate, ethylene glycol methyl ether (meth)acrylamide, mono-2-(methacryloyloxy)ethyl maleate, mono-2-(methacryloyloxy)ethyl succinate, itaconic acid,

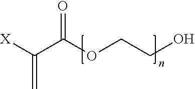

wherein X is hydrogen or methyl, and n is 2, 3, 4, 5, 6, 7, 8, 9, or 10, and combinations thereof; and 2 to 50 mole percent of third units derived from a third monomer selected from the group consisting of C₁-C₆-alkylene bis((meth)acrylate)s, C₁-C₆-alkylene bis((meth)acrylamide)s, ethylene glycol di(meth)acrylate, diethylene glycol di(meth)acrylate, triethylene glycol di(meth)acrylate, tetraethylene glycol di(meth)acrylate, bisphenol A glycerolate di(meth)acrylate, diurethane di(meth)acrylate, trimethylolpropane tri(meth)acrylate, di(trimethylolpropane) tetra(meth)acrylate, polycaprolactone di(meth)acrylate, polycaprolactone tri(meth)acrylate, and combinations thereof; wherein the functionalized crosslinked acrylate copolymer comprises 0.6 to 25 mole percent of reactive functional groups selected from the group consisting of (meth)acrylate, (meth)acrylamido, and combinations thereof; and wherein all mole percent values are based on total moles of first units, second units, and third units in the functionalized crosslinked acrylate copolymer.

Another embodiment is a method of forming a functionalized crosslinked acrylate copolymer, comprising: partially copolymerizing a monomer mixture comprising a first monomer selected from the group consisting of C₁₀-C₂₄-alkyl (meth)acrylates, C₁₀-C₂₄-alkenyl (meth)acrylates, C₁₀-C₂₄-alkyl (meth)acrylamides, C₁₀-C₂₄-alkenyl (meth)acrylamides, and combinations thereof; a second monomer selected from the group consisting of (meth)acrylic acid, C₁-C₈-alkyl (meth)acrylates, C₁-C₈-alkyl (meth)acrylamides, 2-carboxyethyl (meth)acrylate, C₂-C₈-hydroxyalkyl (meth)acrylates, C₂-C₈-hydroxyalkyl (meth)acrylamides, ethylene glycol methyl ether (meth)acrylate, ethylene glycol methyl ether (meth)acrylamide, mono-2-(methacryloyloxy)ethyl maleate, mono-2-(methacryloyloxy)ethyl succinate, itaconic acid,

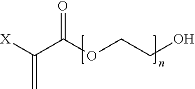

wherein X is hydrogen or methyl, and n is 2, 3, 4, 5, 6, 7, 8, 9, or 10, and combinations thereof; and a third monomer selected from the group consisting of C₁-C₆-alkylene bis((meth)acrylate)s, C₁-C₆-alkylene bis((meth)acrylamide)s, ethylene glycol di(meth)acrylate, diethylene glycol di(meth)acrylate, triethylene glycol di(meth)acrylate, tetraethylene glycol di(meth)acrylate, bisphenol A diglycidyl ether di(meth)acrylate, diurethane di(meth)acrylate, trimethylolpropane tri(meth)acrylate, di(trimethylolpropane) tetra(meth)acrylate, polycaprolactone di(meth)acrylate, polycaprolactone tri(meth)acrylate, and combinations thereof to form a functionalized crosslinked acrylate copolymer comprising 15 to 63 mole percent of first units derived from the first monomer, 35 to 83 mole percent of second units derived from the second monomer, and 2 to 50 mole percent of third units derived from the third monomer; wherein of the 2 to 50 mole percent of third units derived from the third monomer, 0.6 to 25 mole percent have at least one pendant (meth)acryloyloxy or (meth)acrylamido group, and 25 to 49.4 mole percent have zero pendant (meth)acryloyloxy or (meth)acrylamido groups; and wherein all mole percent values are based on total moles of first units, second units, and third units in the functionalized crosslinked acrylate copolymer.

Another embodiment is a curable composition comprising the functionalized crosslinked acrylate copolymer in any of its herein-described variations, and a copolymerizable monomer.

Another embodiment is a cured composition comprising the product of curing a curable composition comprising the functionalized crosslinked acrylate copolymer in any of its herein-described variations, and a copolymerizable monomer.

Another embodiment is a curable composition comprising: an unfunctionalized uncrosslinked acrylate block copolymer comprising a first block and a second block; wherein the first block comprises first units derived from a first monomer selected from the group consisting of C₁₀-C₂₄-alkyl (meth)acrylates, C₁₀-C₂₄-alkenyl (meth)acrylates, C₁₀-C₂₄-alkyl (meth)acrylamides, C₁₀-C₂₄-alkenyl (meth)acrylamides, and combinations thereof; and the second block comprises second units is derived from a second monomer selected from the group consisting of (meth)acrylic acid, C₁-C₈-alkyl (meth)acrylates, C₁-C₈-alkyl (meth)acrylamides, 2-carboxyethyl (meth)acrylate, C₂-C₈-hydroxyalkyl (meth)acrylates, C₂-C₈-hydroxyalkyl (meth)acrylamides, ethylene glycol methyl ether (meth)acrylate, ethylene glycol methyl ether (meth)acrylamide, mono-2-(methacryloyloxy)ethyl maleate, mono-2-(methacryloyloxy)ethyl succinate, itaconic acid,

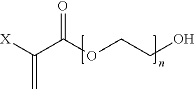

wherein X is hydrogen or methyl, and n is 2, 3, 4, 5, 6, 7, 8, 9, or 10, and combinations thereof; wherein the unfunctionalized uncrosslinked acrylate block copolymer comprises 15 to 65 mole percent of first units and 35 to 85 mole percent of second units, based on the total mole percent of first units and second units in the unfunctionalized uncrosslinked acrylate block copolymer; and a polymerizable monomer comprising a sixth monomer comprising two or more reactive groups selected from the group consisting of (meth)acryloyloxy groups, (meth)acrylamido groups, and combinations thereof.

Another embodiment is a cured composition comprising the product of curing the curable composition comprising the unfunctionalized uncrosslinked acrylate block copolymer, and the polymerizable monomer comprising the sixth monomer.

These and other embodiments are described in detail below.

### BRIEF DESCRIPTION OF THE DRAWINGS

FIG. 1 shows chemical structures for monomers, a solvent, and an initiator used to generate functionalized copolymers and/or adhesive compositions; included are structures for bisphenol A glycerolate dimethacrylate (Bis-GMA), tetraethyleneglycol dimethacrylate (TEGDMA), N,N-dimethylacrylamide (DMAA), 2-hydroxyethyl methacrylate (HEMA), methyl methacrylate (MMA), ethanol (EtOH), and 2,2-dimethoxy-2-phenylacetophenone (DMPA).

FIG. 2 is a schematic representation of a synthetic procedure for synthesis of a functionalized crosslinked random copolymer by copolymerizing stearyl acrylate (SA; 1) with N,N-dimethylacrylamide (DMAA; 2) or 2-ethylhexyl acrylate (EHA; 4) or acrylic acid (AA; 5), in the presence of the crosslinking agent N,N′-methylenebisacrylamide (MBAA; 3); the copolymerization is terminated at 70% double bond conversion.

FIG. 3(a) is a plot of tan delta versus temperature as a function of whether the functionalized copolymer, derived from copolymerization of stearyl acrylate, acrylic acid, and N,N′-methylenebis(acrylamide), is dry (solid line) or wet (dashed line); the inset of FIG. 3(a) is a schematic illustration of self-assembled long-chain n-alkyl side chains in the functionalized copolymer; FIG. 3(b) is an idealized image from Mettler Toledo (not data generated by the present inventors) showing that dynamic mechanical analysis can detect transitions associated with crystallization of side chains from the first monomer.

FIG. 4 is a plot of viscosity (in units of Pascal-seconds) at 25° C. versus content of the functionalized copolymer poly(SA-co-DMAA2575) (in units of weight percent) in a 2:1 weight ratio solution of bisphenol A glycerolate dimethacrylate (BisGMA) and ethanol.

FIG. 5(a) is a plot of percent conversion versus photopolymerization time as a function of weight percent of added functionalized copolymer for a tetraethylene glycol dimethacrylate/N,N-dimethylacrylamide mixture; FIG. 5(b) is a plot of storage modulus and tan delta versus temperature as a function of weight percent of added functionalized copolymer.

FIG. 6 is an image of adhesive compositions photopolymerized in the absence of water (samples 1 and 3) and the presence of water (samples 2 and 4); samples 1 and 2 do not include functionalized copolymer; samples 3 and 4 include 2.5 weight percent of a functionalized copolymer prepared from a monomer mixture comprising stearyl acrylate and N,N-dimethylacrylamide (in a 50:50 mole ratio), and 0.03 M N,N′-methylenebisacrylamide; sample 2, without functionalized copolymer and polymerized in the presence of water, did not yield a coherent film.

FIG. 7 is a bar graph of flexural strength (in megapascals) as a function of sample type, where the samples are, from left to right, a control with no functionalized copolymer, a sample with 1 weight percent of a functionalized crosslinked random copolymer of stearyl acrylate and 2-ethylhexyl acrylate in a 25:75 mole ratio, and a sample with 1 weight percent of an unfunctionalized uncrosslinked diblock copolymer of stearyl acrylate and 2-ethylhexyl acrylate in a 1:1 mole ratio.

FIG. 8 is an illustrative synthesis of an acrylated phenolic azobenzene monomer, where a phenolic azobenzene reacts with acryloyl chloride in the presence of trimethylamine in dichloromethane solvent at room temperature under a nitrogen atmosphere to form the acrylated phenolic azobenzene monomer.

FIG. 9 is a stress versus strain curve for a Type I Reactive Polymer (Type I RP) and a control polymer.

FIG. 10 is a bar chart of wet flexural strength as a function of composition for four compositions: a control polymer composition (labeled “Control”), a composition labeled “Component 1” that comprises 90 weight percent of the control polymer composition and 10 weight percent of a Component 1 reactive polymer, a composition labeled “Component 2” that comprises 90 weight percent of the control polymer composition and 10 weight percent of a Component 2 reactive polymer, and a composition labeled “RP-Pair2” that comprises 90 weight percent of the control polymer composition, 5 weight percent of the Component 1 reactive polymer, and 5 weight percent of the Component 2 reactive polymer.

FIG. 11 is a bar chart of cell viability (in units of percent) as a function of eluate concentration and composition.

FIG. 12 is a bar chart of viscosity (in units of millipascal-seconds) as a function of composition.

FIG. 13 is a bar chart of peak stress (in units of megapascals) as a function of composition after exposure to a sucrose-rich broth for 72 hours; the inset is a photographic image of the experimental apparatus.

### DETAILED DESCRIPTION OF THE INVENTION

The present inventors have determined that addition of a particular functionalized crosslinked acrylate copolymer to a curable composition provides increased strength both immediately after curing of the composition and over time as the cured composition is exposed to water and/or other polar solvents. This is in contrast to the same curable composition without the functionalized crosslinked acrylate copolymer, which is weaker immediately after curing and becomes weaker still on exposure to water. The adhesive composition with the functionalized crosslinked acrylate copolymer thus is expected to contribute to more durable and longer-lasting dental restorations. The functionalized crosslinked acrylate copolymer can also compatibilize a curable composition comprising the functionalized crosslinked acrylate copolymer and a copolymerizable monomer.

One embodiment is a functionalized crosslinked acrylate copolymer comprising: 15 to 63 mole percent of first units derived from a first monomer selected from the group consisting of C₁₀-C₂₄-alkyl (meth)acrylates, C₁₀-C₂₄-alkenyl (meth)acrylates, C₁₀-C₂₄-alkyl (meth)acrylamides, C₁₀-C₂₄-alkenyl (meth)acrylamides, and combinations thereof; 35 to 83 mole percent of second units derived from a second monomer selected from the group consisting of (meth)acrylic acid, C₁-C₈-alkyl (meth)acrylates, C₁-C₈-alkyl (meth)acrylamides, 2-carboxyethyl (meth)acrylate, C₂-C₈-hydroxyalkyl (meth)acrylates, C₂-C₈-hydroxyalkyl (meth)acrylamides, ethylene glycol methyl ether (meth)acrylate, ethylene glycol methyl ether (meth)acrylamide, mono-2-(methacryloyloxy)ethyl maleate, mono-2-(methacryloyloxy)ethyl succinate, itaconic acid,

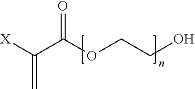

wherein X is hydrogen or methyl, and n is 2, 3, 4, 5, 6, 7, 8, 9, or 10, and combinations thereof; and 2 to 50 mole percent of third units derived from a third monomer selected from the group consisting of C₁-C₆-alkylene bis((meth)acrylate)s, C₁-C₆-alkylene bis((meth)acrylamide)s, ethylene glycol di(meth)acrylate, diethylene glycol di(meth)acrylate, triethylene glycol di(meth)acrylate, tetraethylene glycol di(meth)acrylate, bisphenol A glycerolate di(meth)acrylate, diurethane di(meth)acrylate, trimethylolpropane tri(meth)acrylate, di(trimethylolpropane) tetra(meth)acrylate, polycaprolactone di(meth)acrylate, polycaprolactone tri(meth)acrylate, and combinations thereof; wherein the functionalized crosslinked acrylate copolymer comprises 0.6 to 25 mole percent of reactive functional groups selected from the group consisting of (meth)acrylate, (meth)acrylamido, and combinations thereof; and wherein all mole percent values are based on total moles of first units, second units, and third units in the functionalized crosslinked acrylate copolymer.

In the context of the term “functionalized crosslinked acrylate copolymer,” “functionalized” means that the copolymer comprises reactive (polymerizable) functional groups, preferably (meth)acryloyl groups (in the form of, for example, acrylate groups, methacrylate groups, acrylamide groups, methacrylamide groups, or a combination thereof). As described below, the reactive functional groups can be derived from (a) a fraction of the third monomer in which some but not all reactive functional groups of the third monomer are incorporated into the copolymer backbone; (b) post-polymerization functionalization of pendant hydroxyl groups to form reactive functional groups; or a combination of (a) and (b).

Also in the context of the term “functionalized crosslinked acrylate copolymer,” “crosslinked” means that the copolymer comprises the residue of a third monomer in which at least two functional groups of the third monomer have been incorporated into the backbone of the copolymer.

Also in the context of the term “functionalized crosslinked acrylate copolymer,” “acrylate copolymer” means that the copolymer comprises the residue of at least two types of monofunctional (meth)acryloyl monomers, the first being the “first monomer” (containing a long-chain alkyl or alkenyl group) and the second being the “second monomer” (not containing a long-chain alkyl or alkenyl group).

The “crystallization-driven self-strengthening” properties of the copolymer are derived, at least in part, from the formation of semicrystalline domains derived from the long hydrocarbon chain of the first monomer. Partial formation of the semicrystalline domains occurs during curing (polymerization) of a composition comprising the functionalized copolymer (for example, during formation of a dental restoration comprising the copolymer). Additional formation of the semicrystalline domains occurs during long-term exposure to water (for example, during exposure of the dental restoration to water in the oral cavity).

As used herein, the prefix “(meth)acryl-” means “acryl-” or “methacryl-”. For example, “(meth)acrylate” means “acrylate” or “methacrylate”.

The terms “(meth)acrylate” and “(meth)acryloyloxy” are used interchangeably to denote a monovalent substituent having the structure—O—C(═O)—C(X)═CH₂, wherein X is hydrogen or methyl.

The terms “(meth)acrylamide” and “(meth)acrylamido” are used interchangeably to denote a monovalent substituent having the structure—N(H)—C(═O)—C(X)═CH₂, wherein X is hydrogen or methyl.

The term “(meth)acryloyl” denotes a monovalent substituent having the structure—C(═O)—C(X)═CH₂, wherein X is hydrogen or methyl. A (meth)acryloyl group is part of the structure of a (meth)acrylate group, a (meth)acryloyloxy group, a (meth)acrylamide group, and a (meth)acrylamido group.

The first monomer is selected from the group consisting of C₁₀-C₂₄-alkyl (meth)acrylates, C₁₀-C₂₄-alkenyl (meth)acrylates, C₁₀-C₂₄-alkyl (meth)acrylamides, C₁₀-C₂₄-alkenyl (meth)acrylamides, and combinations thereof. Examples of C₁₀-C₂₄-alkyl (meth)acrylates include decyl acrylate, decyl methacrylate, lauryl acrylate, lauryl methacrylate, myristyl acrylate, myristyl methacrylate, cetyl acrylate, cetyl methacrylate, stearyl acrylate, stearyl methacrylate, arachidyl acrylate, arachidyl methacrylate, behenyl acrylate, behenyl methacrylate, lignoceryl acrylate, lignoceryl methacrylate, and combinations thereof. Examples of C₁₀-C₂₄-alkyl (meth)acrylates include decyl acrylate, decyl methacrylate, lauryl acrylate, lauryl methacrylate, myristyl acrylate, myristyl methacrylate, cetyl acrylate, cetyl methacrylate, stearyl acrylate, stearyl methacrylate, arachidyl acrylate, arachidyl methacrylate, behenyl acrylate, behenyl methacrylate, lignoceryl acrylate, lignoceryl methacrylate, and combinations thereof. Examples of C₁₀-C₂₄-alkenyl (meth)acrylates include 10-undecenyl acrylate, 10-undecenyl methacrylate, palmitoleyl acrylate, palmitoleyl methacrylate, oleyl acrylate, oleyl methacrylate, erucyl acrylate, erucyl methacrylate, and combination thereof. Examples of C₁₀-C₂₄-alkyl (meth)acrylamides include N-decyl acrylamide, N-decyl methacrylamide, N-lauryl acrylamide, N-lauryl methacrylamide, N-myristyl acrylamide, N-myristyl methacrylamide, N-cetyl acrylamide, N-cetyl methacrylamide, N-stearyl acrylamide, N-stearyl methacrylamide, N-arachidyl acrylamide, N-arachidyl methacrylamide, N-behenyl acrylamide, N-behenyl methacrylamide, N-lignoceryl acrylamide, N-lignoceryl methacrylamide, and combinations thereof. Examples of C₁₀-C₂₄-alkenyl (meth)acrylamides include N-10-undecenyl acrylamide, N-10-undecenyl methacrylamide, N-palmitoleyl acrylamide, N-palmitoleyl methacrylamide, N-oleyl acrylamide, N-oleyl methacrylamide, N-erucyl acrylamide, N-erucyl methacrylamide, and combinations thereof.

In some embodiments, the first monomer is a C₁₂-C₂₂-alkyl (meth)acrylate. In some embodiments, the first monomer is stearyl (meth)acrylate.

The functionalized crosslinked acrylate copolymer comprises 15 to 63 mole percent of first units derived from the first monomer, based on total moles of first units, second units, and third units in the functionalized crosslinked acrylate copolymer. Within this range, the mole percent of first units derived from the first monomer can be 20 to 60 mole percent, or 20 to 55 mole percent.

The second monomer is selected from the group consisting of (meth)acrylic acid, C₁-C₈-alkyl (meth)acrylates, C₁-C₈-alkyl (meth)acrylamides, 2-carboxyethyl (meth)acrylate, C₂-C₈-hydroxyalkyl (meth)acrylates (including, for example, 2-hydroxyethyl (meth)acrylate), C₂-C₈-hydroxyalkyl (meth)acrylamides (including, for example, 2-hydroxypropyl (meth)acrylamide, N-hydroxyethyl (meth)acrylamide, and N-tris(hydroxymethyl)methyl (meth)acrylamide), ethylene glycol methyl ether (meth)acrylate, ethylene glycol methyl ether (meth)acrylamide, mono-2-(methacryloyloxy)ethyl maleate (e.g., CAS Reg. No. 51978-15-5), mono-2-(methacryloyloxy)ethyl succinate (e.g., CAS Reg. No. 20882-04-6), itaconic acid,

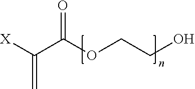

wherein X is hydrogen or methyl, and n is 2, 3, 4, 5, 6, 7, 8, 9, or 10, and combinations thereof. Examples of C₁-C₈-alkyl (meth)acrylates include ethyl acrylate, ethyl methacrylate, butyl acrylate, butyl methacrylate, hexyl acrylate, hexyl methacrylate, octyl acrylate, octyl methacrylate, 2-ethylhexyl acrylate, 2-ethylhexyl methacrylate, and combinations thereof. Examples of C₁-C₈-alkyl (meth)acrylamides include, for example, N-ethyl acrylamide, N-ethyl methacrylamide, N-iso-propyl acrylamide, N-iso-propyl methacrylamide, N-butyl acrylamide, N-butyl methacrylamide, N-tert-butyl acrylamide, N-tert-butyl methacrylamide, N-hexyl acrylamide, N-hexyl methacrylamide, N-octyl acrylamide, N-octyl methacrylamide, N-2-ethylhexyl acrylamide, N-2-ethylhexyl methacrylamide, N,N-dimethyl acrylamide, and combinations thereof. Examples of C₂-C₈-hydroxyalkyl (meth)acrylates include 2-hydroxyethyl acrylate, 2-hydroxyethyl methacrylate, 2-hydroxypropyl acrylate, 2-hydroxypropyl methacrylate, tris(hydroxymethyl)methyl acrylate, tris(hydroxymethyl)methyl methacrylate, and combinations thereof. Examples of C₂-C₈-hydroxyalkyl (meth)acrylamides include N-2-hydroxyethyl (meth)acrylamide, N,N-bis(2-hydroxyethyl) (meth)acrylamide, 2-hydroxypropyl (meth)acrylamide, N,N,N-tris(hydroxymethyl)methyl (meth)acrylamide, and combinations thereof.

In some embodiments, the second monomer is selected from the group consisting of C₁-C₈-alkyl (meth)acrylates, C₁-C₈-alkyl (meth)acrylamides, 2-carboxyethyl (meth)acrylate, C₂-C₈-hydroxyalkyl (meth)acrylates, C₂-C₈-hydroxyalkyl (meth)acrylamides, ethylene glycol methyl ether (meth)acrylate, ethylene glycol methyl ether (meth)acrylamide, mono-2-(methacryloyloxy)ethyl maleate, mono-2-(methacryloyloxy)ethyl succinate, itaconic acid,

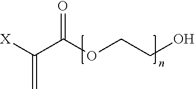

wherein X is hydrogen or methyl, and n is 2, 3, 4, 5, 6, 7, 8, 9, or 10, and combinations thereof.

In some embodiments, the second monomer is selected from the group consisting of C₁-C₈-alkyl (meth)acrylates, C₁-C₈-alkyl (meth)acrylamides, 2-carboxyethyl (meth)acrylate, C₂-C₈-hydroxyalkyl (meth)acrylates, C₂-C₈-hydroxyalkyl (meth)acrylamides, ethylene glycol methyl ether (meth)acrylate, ethylene glycol methyl ether (meth)acrylamide, mono-2-(methacryloyloxy)ethyl maleate, mono-2-(methacryloyloxy)ethyl succinate, itaconic acid,

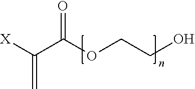

wherein X is hydrogen or methyl, and n is 2, 3, 4, 5, 6, 7, 8, 9, or 10, and combinations thereof, provided that if the second monomer comprises a C₁-C₈-alkyl (meth)acrylamide, it excludes N,N-dimethylacrylamide.

In some embodiments, the second monomer is acrylic acid, ethyl acrylate, butyl acrylate, 2-ethylhexyl acrylate, 2-carboxyethyl acrylate, ethylene glycol methyl ether acrylate, N,N-dimethyl acrylamide, N-iso-propyl acrylamide, N-tert-butyl acrylamide, N-hydroxyethyl acrylamide, 2-hydroxypropyl methacrylamide, N-[tris(hydroxymethyl)methyl]acrylamide,

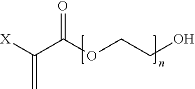

wherein X is hydrogen or methyl, and n is 2, 3, 4, 5, 6, 7, 8, 9, or 10, and combinations thereof.

The functionalized crosslinked acrylate copolymer comprises 35 to 83 mole percent of second units derived from the second monomer, based on total moles of first units, second units, and third units in the functionalized crosslinked acrylate copolymer. Within this range, the mole percent of second units derived from the second monomer can be 40 to 80 mole percent, or 45 to 80 mole percent.

The third monomer is selected from the group consisting of C₁-C₆-alkylene bis((meth)acrylate)s, C₁-C₆-alkylene bis((meth)acrylamide)s, ethylene glycol di(meth)acrylate, diethylene glycol di(meth)acrylate, triethylene glycol di(meth)acrylate, tetraethylene glycol di(meth)acrylate, bisphenol A glycerolate di(meth)acrylate, diurethane di(meth)acrylate (e.g., CAS Reg. No. 72869-86-4), trimethylolpropane tri(meth)acrylate, di(trimethylolpropane) tetra(meth)acrylate (e.g., CAS Reg. No. 94108-97-1), polycaprolactone di(meth)acrylate, polycaprolactone tri(meth)acrylate, and combinations thereof. Examples of C₁-C₆-alkylene bis((meth)acrylate)s include 1,2-ethylene bis((meth)acrylate) (also known as ethylene glycol di(meth)acrylate), 1,3-butylene bis(meth)acrylate) (also known as 1,3-butanediol di(meth)acrylate), 1,4-butylene bis((meth)acrylate) (also known as 1,4-butanediol di(meth)acrylate), 1,6-hexylene bis((meth)acrylate) (also known as 1,6-hexanediol di(meth)acrylate), and combinations thereof. Examples of C₁-C₆-alkylene bis((meth)acrylamide)s include N,N′-methylenebis((meth)acrylamide), N,N′-ethylene bis((meth)acrylamide), N,N′-butylene bis((meth)acrylamide), N,N′-hexylene bis((meth)acrylamide), and combinations thereof. Examples of diethylene glycol di(meth)acrylates include diethylene glycol diacrylate, diethylene glycol dimethacrylate, or a combination thereof. Examples of triethylene glycol di(meth)acrylates include triethylene glycol diacrylate, triethylene glycol dimethacrylate, and combinations thereof. Examples of tetraethylene glycol di(meth)acrylates include tetraethylene glycol diacrylate, tetraethylene glycol dimethacrylate, and combinations thereof. Examples of bisphenol A diglycidyl ether di(meth)acrylates include bisphenol A diglycidyl ether diacrylate, bisphenol A diglycidyl ether dimethacrylate, and combinations thereof. Additional third monomers include the di-, tri-, and poly(meth)acrylate-functionalized monomers available under the tradename EBECRYL from allnex.

In some embodiments, the third monomer is N,N′-methylenebis((meth)acrylamide), 1,4-butanediol di(meth)acrylate, 1,6-hexanediol di(meth)acrylate, ethylene glycol di(meth)acrylate, diethylene glycol di(meth)acrylate, triethylene glycol di(meth)acrylate, or a combination thereof.

The functionalized crosslinked acrylate copolymer comprises 2 to 50 mole percent of third units derived from the third monomer, based on total moles of first units, second units, and third units in the functionalized crosslinked acrylate copolymer. Within this range, the mole percent of third units derived from the second monomer can be 2 to 35 mole percent, or 2 to 20 mole percent.

The functionalized crosslinked acrylate copolymer comprises reactive functional groups selected from the group consisting of (meth)acrylate (—O—C(═O)—C(X)═CH₂ where X═—H or —CH₃), (meth)acrylamido (—N(H)—C(═O)—C(X)═CH₂ where X═—H or —CH₃), and combinations thereof. The reactive functional groups can be derived from (a) a fraction of the third monomer in which some but not all reactive functional groups of the monomer are incorporated into the copolymer backbone; (b) post-polymerization functionalization of pendant hydroxyl groups to form reactive functional groups; or a combination of (a) and (b). An example of reactive functional groups derived from the third monomer would be if the third monomer comprised N,N′-methylenebis(acrylamide), copolymerization of the first, second, and third monomers was conducted to 70% consumption of double bonds, and a portion of the initial N,N′-methylenebis(acrylamide) reacted so that one of the two acrylamido groups was incorporated into the copolymer backbone and the remaining acrylamido group was unreacted and pendant from the copolymer backbone. An example of reactive functional groups derived from post-polymerization functionalization of pendant hydroxyl groups would be if the second monomer comprised N-hydroxyethyl acrylamide, copolymerization of the first, second, and third monomers therefore led to a copolymer with pendant hydroxyl groups derived from the N-hydroxyethyl acrylamide, and at least a portion of those pendant hydroxyl groups were reacted with a reagent capable of producing a pendant (meth)acrylate group. Examples of reagents capable of producing a pendant (meth)acrylate group include 2-isocyanatoethyl (meth)acrylate (which forms a pendant N-(2-(meth)acryloylethyl) carbamate (CH₂═C(X)C(O)O(CH₂)₂NHC(O)O—) group, wherein X is —H or —CH₃), (meth)acryloyl chloride, (meth)acrylic acid, (meth)acrylic anhydride, and combinations thereof.

The functionalized crosslinked acrylate copolymer comprises 0.6 to 25 mole percent of the reactive functional groups, based on total moles of first units, second units, and third units in the functionalized crosslinked acrylate copolymer. Within this range, the mole percent of reactive functional groups can be 1 to 20 mole percent, or 2 to 20 mole percent.

In some embodiments, the functionalized crosslinked acrylate copolymer is a functionalized crosslinked acrylate block copolymer comprising at least one block comprising first units and at least one block comprising second units. The functionalized crosslinked acrylate block copolymer can be a diblock (AB) copolymer, a triblock (ABA, BAB) copolymer, a tetrablock (ABAB) copolymer, or a pentablock (ABABA, BABAB) copolymer. In some embodiments, the functionalized crosslinked acrylate block copolymer is a diblock (AB) copolymer or a triblock (ABA, BAB) copolymer. In some embodiments, the functionalized crosslinked acrylate block copolymer is a diblock (AB) copolymer.

The functionalized crosslinked acrylate block copolymer can be synthesized by acrylate block copolymer preparation methods known in the art, including Reversible Addition Fragmentation Chain Transfer (RAFT).

In some embodiments, wherein the functionalized crosslinked acrylate copolymer is a functionalized crosslinked acrylate random copolymer comprising randomly dispersed first units and second units. Illustrative syntheses of such polymers are included in the working examples below.

In some embodiments, the functionalized crosslinked acrylate copolymer further comprises 0.5 to 5 mole percent of hydroxy-functionalized azobenzene groups, based on total moles of first units, second units, and third units in the functionalized crosslinked acrylate copolymer. Within this range, the mole percent of hydroxy-functionalized azobenzene groups can be 0.5 to 3 mole percent, or 0.5 to 2 mole percent. In some embodiments, the hydroxy-functionalized azobenzene groups are created by copolymerizing the first, second, and third monomers with an azobenzene-functionalized monomer having the structure

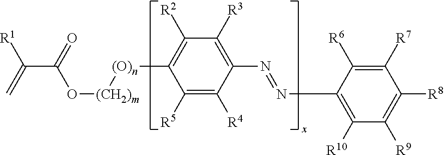

wherein m is an integer from 0 to 20; n is zero or 1, provided that when n is 1, m is an integer from 1 to 20, or 2 to 20; x is an integer from 1 to 10; R¹ is hydrogen or methyl; and R²-R¹⁰ are each independently hydrogen, methyl, or hydroxyl, provided that at least one of R²-R¹⁰ is hydroxyl. FIG. 8 is a model synthesis of an acrylated phenolic azobenzene monomer in which phenolic azobenzene reacts with acryloyl chloride in the presence of trimethylamine to form the acrylated phenolic azobenzene monomer.

In some embodiments, the functionalized crosslinked acrylate copolymer has a weight average molecular weight (M_{w}) of 5 to 1,000 kilodaltons/mole. Within this range, the weight average molecular weight of the functionalized crosslinked acrylate copolymer can be 20 to 800 kilodaltons/mole, or 100 to 600 kilodaltons/mole. In some embodiments, the functionalized crosslinked acrylate copolymer has a polydispersity index of 1 to 3. Polydispersity index, which is unitless, is the ratio of weight average molecular weight (M_{w}) to number average molecular weight (Mₙ). Number average molecular weight (Mₙ), weight average molecular weight (M_{w}), and polydispersity index (PDI=M_{w}/Mₙ) can be determined for random and block copolymers by gel permeation chromatography using a series of four columns spanning molecular weights of 10⁴ to 10⁷ grams/mole, tetrahydrofuran eluent, a flow rate of 1 milliliter/minute, poly(methyl methacrylate) molecular weight standards, and light scattering detection.

In a very specific embodiment, the functionalized crosslinked acrylate copolymer comprises 20 to 50 mole percent of the first units; wherein the first monomer is selected from the group consisting of C₁₂-C₂₂-alkyl (meth)acrylates, and combinations of C₁₂-C₂₂-alkyl (meth)acrylates; 45 to 75 mole percent of the second units; wherein the second monomer is selected from the group consisting of acrylic acid, ethyl acrylate, butyl acrylate, 2-ethylhexyl acrylate, 2-carboxyethyl acrylate, ethylene glycol methyl ether acrylate, N,N-dimethyl acrylamide, N-iso-propyl acrylamide, N-tert-butyl acrylamide, N-hydroxyethyl acrylamide, 2-hydroxypropyl methacrylamide, N-[tris(hydroxymethyl)methyl]acrylamide,

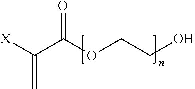

wherein X is hydrogen or methyl, and n is 2, 3, 4, 5, 6, 7, 8, 9, or 10, and combinations thereof; and 5 to 35 mole percent of the third units; wherein the third monomer is selected from the group consisting of N,N′-methylenebis((meth)acrylamide), ethylene glycol di(meth)acrylate, 1,4-butanediol di(meth)acrylate, 1,6-hexanediol di(meth)acrylate, diethylene glycol di(meth)acrylate, triethylene glycol di(meth)acrylate, and combinations thereof; wherein the functionalized crosslinked acrylate copolymer comprises 2 to 20 mole percent of the reactive functional groups. Within this very specific embodiment, the second monomer can be selected from the group consisting of C₁-C₈-alkyl (meth)acrylates, C₁-C₈-alkyl (meth)acrylamides, 2-carboxyethyl (meth)acrylate, C₂-C₈-hydroxyalkyl (meth)acrylates, C₂-C₈-hydroxyalkyl (meth)acrylamides, ethylene glycol methyl ether (meth)acrylate, ethylene glycol methyl ether (meth)acrylamide, and combinations thereof. Also within this very specific embodiment, the second monomer can be selected from the group consisting of C₁-C₈-alkyl (meth)acrylates, C₁-C₈-alkyl (meth)acrylamides, 2-carboxyethyl (meth)acrylate, C₂-C₈-hydroxyalkyl (meth)acrylates, C₂-C₈-hydroxyalkyl (meth)acrylamides, ethylene glycol methyl ether (meth)acrylate, ethylene glycol methyl ether (meth)acrylamide, and combinations thereof, provided that if the second monomer comprises a C₁-C₈-alkyl (meth)acrylamide, it excludes N,N-dimethylacrylamide. Also within this very specific embodiment, the functionalized crosslinked acrylate copolymer can be a functionalized crosslinked acrylate random copolymer comprising randomly dispersed first units and second units.

In another very specific embodiment, the functionalized crosslinked acrylate copolymer comprises 15 to 60 mole percent of the first units; wherein the first monomer is selected from the group consisting of C₁₂-C₂₂-alkyl (meth)acrylates, and combinations of C₁₂-C₂₂-alkyl (meth)acrylates; 15 to 60 mole percent of the second units; wherein the second monomer is selected from the group consisting of acrylic acid, ethyl acrylate, butyl acrylate, 2-ethylhexyl acrylate, 2-carboxyethyl acrylate, ethylene glycol methyl ether acrylate, N,N-dimethyl acrylamide, N-iso-propyl acrylamide, N-tert-butyl acrylamide, N-hydroxyethyl acrylamide, 2-hydroxypropyl methacrylamide, N-[tris(hydroxymethyl)methyl]acrylamide, and combinations thereof; and 15 to 35 mole percent of the third units; wherein the third monomer is selected from the group consisting of N,N′-methylenebis((meth)acrylamide), ethylene glycol di(meth)acrylate, 1,4-butanediol di(meth)acrylate, 1,6-hexanediol di(meth)acrylate, diethylene glycol di(meth)acrylate, triethylene glycol di(meth)acrylate, and combinations thereof; wherein the functionalized crosslinked acrylate copolymer comprises 2 to 20 mole percent of the reactive functional groups.

Another embodiment is a method of forming a functionalized crosslinked acrylate copolymer, comprising: partially copolymerizing a monomer mixture comprising a first monomer selected from the group consisting of C₁₀-C₂₄-alkyl (meth)acrylates, C₁₀-C₂₄-alkenyl (meth)acrylates, C₁₀-C₂₄-alkyl (meth)acrylamides, C₁₀-C₂₄-alkenyl (meth)acrylamides, and combinations thereof; a second monomer selected from the group consisting of (meth)acrylic acid, C₁-C₈-alkyl (meth)acrylates, C₁-C₈-alkyl (meth)acrylamides, 2-carboxyethyl (meth)acrylate, C₂-C₈-hydroxyalkyl (meth)acrylates, C₂-C₈-hydroxyalkyl (meth)acrylamides, ethylene glycol methyl ether (meth)acrylate, ethylene glycol methyl ether (meth)acrylamide, mono-2-(methacryloyloxy)ethyl maleate, mono-2-(methacryloyloxy)ethyl succinate, itaconic acid,

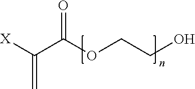

wherein X is hydrogen or methyl, and n is 2, 3, 4, 5, 6, 7, 8, 9, or 10, and combinations thereof; and a third monomer selected from the group consisting of C₁-C₆-alkylene bis((meth)acrylate)s, C₁-C₆-alkylene bis((meth)acrylamide)s, ethylene glycol di(meth)acrylate, diethylene glycol di(meth)acrylate, triethylene glycol di(meth)acrylate, tetraethylene glycol di(meth)acrylate, bisphenol A diglycidyl ether di(meth)acrylate, diurethane di(meth)acrylate, trimethylolpropane tri(meth)acrylate, di(trimethylolpropane) tetra(meth)acrylate, polycaprolactone di(meth)acrylate, polycaprolactone tri(meth)acrylate, and combinations thereof to form a functionalized crosslinked acrylate copolymer comprising 15 to 63 mole percent of first units derived from the first monomer, 35 to 83 mole percent of second units derived from the second monomer, and 2 to 50 mole percent of third units derived from the third monomer; wherein of the 2 to 50 mole percent of third units derived from the third monomer, 0.6 to 25 mole percent have at least one pendant (meth)acryloyloxy or (meth)acrylamido group, and 25 to 49.4 mole percent have zero pendant (meth)acryloyloxy or (meth)acrylamido groups; and wherein all mole percent values are based on total moles of first units, second units, and third units in the functionalized crosslinked acrylate copolymer. In some embodiments, of the 2 to 50 mole percent of third units derived from the third monomer, 0.6 to 25 mole percent have at least one pendant (meth)acryloyloxy (—O—C(═O)—C(X)═CH₂, wherein X is hydrogen or methyl) or (meth)acrylamido group (—NH—C(═O)—C(X)═CH₂, wherein X is hydrogen or methyl), and 25 to 49.4 mole percent have zero pendant (meth)acryloyloxy or (meth)acrylamido groups

In some embodiments of the method, the monomer mixture comprises 15 to 63 mole percent of the first monomer, 35 to 83 mole percent of the second monomer, and 2 to 50 mole percent of the third monomer, based on total moles of first monomer, second monomer, and third monomer in the monomer mixture.

All of the chemical variations described above in the context of the functionalized crosslinked acrylate copolymer apply as well to the method of forming a functionalized crosslinked acrylate copolymer.

In some embodiments of the method, the partially copolymerizing comprises polymerizing 50 to 80 mole percent of polymerizable groups contributed by the first monomer, second monomer, and third monomer. Within this limit, the partially copolymerizing can comprise polymerizing 55 to 75 mole percent, or 60 to 70 mole percent of polymerizable groups contributed by the first monomer, second monomer, and third monomer.

In some embodiments of the method, the second monomer comprises a C₂-C₈-hydroxyalkyl (meth)acrylate, a C₂-C₈-hydroxyalkyl (meth)acrylamide, or a combination thereof; the second units comprise pendant hydroxyl groups; and the method further comprises reacting at least a portion of the pendant hydroxyl groups with a reagent capable of producing a pendant (meth)acrylate group. One example of such a reagent is 2-isocyanatoethyl (meth)acrylate, which reacts with pendant hydroxyl groups to form pendant N-(2-(meth)acryloylethyl) carbamate (CH₂═C(X)C(O)O(CH₂)₂NHC(O)O—) groups, wherein X is —H or —CH₃.

In a very specific embodiment of the method, the functionalized crosslinked acrylate copolymer comprises: 20 to 50 mole percent of the first units; wherein the first monomer is selected from the group consisting of C₁₂-C₂₂-alkyl (meth)acrylates, and combinations of C₁₂-C₂₂-alkyl (meth)acrylates; 45 to 75 mole percent of the second units; wherein the second monomer is selected from the group consisting of acrylic acid, ethyl acrylate, butyl acrylate, 2-ethylhexyl acrylate, 2-carboxyethyl acrylate, ethylene glycol methyl ether acrylate, N,N-dimethyl acrylamide, N-iso-propyl acrylamide, N-tert-butyl acrylamide, N-hydroxyethyl acrylamide, 2-hydroxypropyl methacrylamide, N-[tris(hydroxymethyl)methyl]acrylamide,

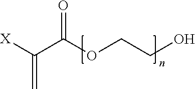

wherein X is hydrogen or methyl, and n is 2, 3, 4, 5, 6, 7, 8, 9, or 10, and combinations thereof; and 5 to 35 mole percent of the third units; wherein the third monomer is selected from the group consisting of N,N′-methylenebis((meth)acrylamide), ethylene glycol di(meth)acrylate, 1,4-butanediol di(meth)acrylate, 1,6-hexanediol di(meth)acrylate, diethylene glycol di(meth)acrylate, triethylene glycol di(meth)acrylate, and combinations thereof; wherein the functionalized crosslinked acrylate copolymer comprises 2 to 20 mole percent of the reactive functional groups. Within this very specific embodiment, the second monomer can be selected from the group consisting of C₁-C₈-alkyl (meth)acrylates, C₁-C₈-alkyl (meth)acrylamides, 2-carboxyethyl (meth)acrylate, C₂-C₈-hydroxyalkyl (meth)acrylates, C₂-C₈-hydroxyalkyl (meth)acrylamides, ethylene glycol methyl ether (meth)acrylate, ethylene glycol methyl ether (meth)acrylamide, and combinations thereof. Also within this very specific embodiment, the second monomer can be selected from the group consisting of C₁-C₈-alkyl (meth)acrylates, C₁-C₈-alkyl (meth)acrylamides, 2-carboxyethyl (meth)acrylate, C₂-C₈-hydroxyalkyl (meth)acrylates, C₂-C₈-hydroxyalkyl (meth)acrylamides, ethylene glycol methyl ether (meth)acrylate, ethylene glycol methyl ether (meth)acrylamide, and combinations thereof, provided that if the second monomer comprises a C₁-C₈-alkyl (meth)acrylamide, it excludes N,N-dimethylacrylamide. Also with this very specific embodiment, the functionalized crosslinked acrylate copolymer can be a functionalized crosslinked acrylate random copolymer comprising randomly dispersed first units and second units.

Another embodiment is a curable composition comprising: the functionalized crosslinked acrylate copolymer in any of its variations described herein, and a copolymerizable monomer. Uses of the curable composition include dental adhesive compositions and dental restorative compositions. The copolymerizable monomer can be monofunctional (i.e., having one functional group copolymerizable with the reactive functional groups of the functionalized crosslinked acrylate copolymer), difunctional (i.e., having two functional groups copolymerizable with the reactive functional groups of the functionalized crosslinked acrylate copolymer), trifunctional (i.e., having three functional groups copolymerizable with the reactive functional groups of the functionalized crosslinked acrylate copolymer), polyfunctional (i.e., having four or more functional groups copolymerizable with the reactive functional groups of the functionalized crosslinked acrylate copolymer), or a combination thereof. In some embodiments, the copolymerizable monomer comprises a monofunctional monomer. In some embodiments, the copolymerizable monomer comprises a difunctional monomer, a trifunctional monomer, a polyfunctional monomer, or a combination thereof. In some embodiments, the copolymerizable monomer comprises a monofunctional monomer and a difunctional monomer or a trifunctional monomer or a polyfunctional monomer, or a combination thereof.

Monofunctional copolymerizable monomers include those described in the context of the first and second monomers used to form the functionalized crosslinked acrylate copolymer. Difunctional, trifunctional, and polyfunctional copolymerizable monomers include those described in the context of the third monomer used to form the functionalized crosslinked acrylate copolymer. In some embodiments, the copolymerizable monomer comprises at least one hydroxyl group or at least one carboxylic acid groups. Examples of such monomers include 2-carboxyethyl (meth)acrylate, C₂-C₈-hydroxyalkyl (meth)acrylates, C₂-C₈-hydroxyalkyl (meth)acrylamides, ethylene glycol methyl ether (meth)acrylate, ethylene glycol methyl ether (meth)acrylamide, mono-2-(methacryloyloxy)ethyl maleate, mono-2-(methacryloyloxy)ethyl succinate, itaconic acid,

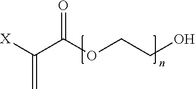

wherein X is hydrogen or methyl, and n is 2, 3, 4, 5, 6, 7, 8, 9, or 10, and combinations thereof.

In some embodiments, the curable composition comprises 2 to 25 weight percent of the functionalized crosslinked acrylate copolymer, and 75 to 98 weight percent of the copolymerizable monomer, based on the total weight of the curable composition. Within the range of 2 to 25 weight percent, the functionalized crosslinked acrylate copolymer content of the curable composition can be 3 to 20 weight percent, or 5 to 15 weight percent. Within the range of 75 to 98 weight percent, the copolymerizable monomer content of the curable composition can be 80 to 97 weight percent, or 85 to 95 weight percent.

In some embodiments of the curable composition, the functionalized crosslinked acrylate copolymer comprises a first functionalized crosslinked acrylate copolymer comprising second units derived from a second monomer selected from the group consisting of C₂-C₈-hydroxyalkyl (meth)acrylates, C₂-C₈-hydroxyalkyl (meth)acrylamides,

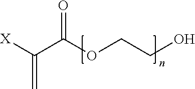

wherein X is hydrogen or methyl, and n is 2, 3, 4, 5, 6, 7, 8, 9, or 10, and combinations thereof; and a second functionalized crosslinked acrylate copolymer comprising second units derived from a second monomer selected from the group consisting of (meth)acrylic acid, mono-2-(methacryloyloxy)ethyl maleate, mono-2-(methacryloyloxy)ethyl succinate, itaconic acid, and combinations thereof; wherein the curable composition comprises, based on the total weight of the curable composition, 2 to 15 weight percent of the first functionalized crosslinked acrylate copolymer and 2 to 15 weight percent of the second functionalized crosslinked acrylate copolymer. Within the range of 2 to 15 weight percent, the weight percent of the first functionalized crosslinked acrylate copolymer in the curable composition can be 3 to 12 weight percent, or 4 to 8 weight percent. Within the range of 2 to 15 weight percent, the weight percent of the second functionalized crosslinked acrylate copolymer in the curable composition can be 3 to 12 weight percent, or 4 to 8 weight percent. In some embodiments of this curable composition, it comprises 5 to 15 weight percent total, or 7 to 13 weight percent total, of the first functionalized crosslinked acrylate copolymer and the second functionalized crosslinked acrylate copolymer, based on the total weight of the curable composition.

In a specific embodiment, the curable composition comprises, based on the total weight of the curable composition, 2 to 25 weight percent of the functionalized crosslinked acrylate copolymer, and 75 to 98 weight percent of the copolymerizable monomer; wherein the copolymerizable monomer comprises, based on 100 parts by weight of the copolymerizable monomer, 80 to 95 parts by weight of a fourth monomer selected from the group consisting of (meth)acrylic acid, C₁-C₈-alkyl (meth)acrylates, C₁-C₈-alkyl (meth)acrylamides, 2-carboxyethyl (meth)acrylate, C₂-C₈-hydroxyalkyl (meth)acrylates, C₂-C₈-hydroxyalkyl (meth)acrylamides, ethylene glycol methyl ether (meth)acrylate, ethylene glycol methyl ether (meth)acrylamide, mono-2-(methacryloyloxy)ethyl maleate, mono-2-(methacryloyloxy)ethyl succinate, itaconic acid,

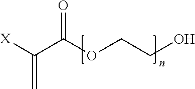

wherein X is hydrogen or methyl, and n is 2, 3, 4, 5, 6, 7, 8, 9, or 10, and combinations thereof; and 5 to 20 parts by weight of a fifth monomer selected from the group consisting of diethyleneglycol di(meth)acrylate, triethyleneglycol di(meth)acrylate, tetraethyleneglycol di(meth)acrylate, bisphenol A glycerolate di(meth)acrylate, diurethane di(meth)acrylate, C₁-C₆-alkylene bis((meth)acrylate)s, C₁-C₆-alkylene bis((meth)acrylamide)s, trimethylolpropane tri(meth)acrylate, di(trimethylolpropane) tetra(meth)acrylate, polycaprolactone di(meth)acrylate, polycaprolactone tri(meth)acrylate, and combinations thereof.

In another very specific embodiment of the curable composition, the functionalized crosslinked acrylate copolymer comprises: 15 to 60 mole percent of the first units; wherein the first monomer is selected from the group consisting of C₁₂-C₂₂-alkyl (meth)acrylates, and combinations of C₁₂-C₂₂-alkyl (meth)acrylates; 15 to 60 mole percent of the second units; wherein the second monomer is selected from the group consisting of acrylic acid, ethyl acrylate, butyl acrylate, 2-ethylhexyl acrylate, 2-carboxyethyl acrylate, ethylene glycol methyl ether acrylate, N,N-dimethyl acrylamide, N-iso-propyl acrylamide, N-tert-butyl acrylamide, N-hydroxyethyl acrylamide, 2-hydroxypropyl methacrylamide, N-[tris(hydroxymethyl)methyl]acrylamide,

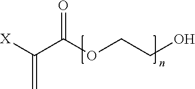

wherein X is hydrogen or methyl, and n is 2, 3, 4, 5, 6, 7, 8, 9, or 10, and combinations thereof; and 15 to 35 mole percent of the third units; wherein the third monomer is selected from the group consisting of N,N′-methylenebis((meth)acrylamide), ethylene glycol di(meth)acrylate, 1,4-butanediol di(meth)acrylate, 1,6-hexanediol di(meth)acrylate, diethylene glycol di(meth)acrylate, triethylene glycol di(meth)acrylate, and combinations thereof; wherein the functionalized crosslinked acrylate copolymer comprises 2 to 20 mole percent of the reactive functional groups. Within this very specific embodiment, the second monomer can be selected from the group consisting of C₁-C₈-alkyl (meth)acrylates, C₁-C₈-alkyl (meth)acrylamides, 2-carboxyethyl (meth)acrylate, C₂-C₈-hydroxyalkyl (meth)acrylates, C₂-C₈-hydroxyalkyl (meth)acrylamides, ethylene glycol methyl ether (meth)acrylate, ethylene glycol methyl ether (meth)acrylamide, and combinations thereof. Also within this very specific embodiment, the second monomer can be selected from the group consisting of C₁-C₈-alkyl (meth)acrylates, C₁-C₈-alkyl (meth)acrylamides, 2-carboxyethyl (meth)acrylate, C₂-C₈-hydroxyalkyl (meth)acrylates, C₂-C₈-hydroxyalkyl (meth)acrylamides, ethylene glycol methyl ether (meth)acrylate, ethylene glycol methyl ether (meth)acrylamide, and combinations thereof, provided that if the second monomer comprises a C₁-C₈-alkyl (meth)acrylamide, it excludes N,N-dimethylacrylamide. Also within this very specific embodiment, the functionalized crosslinked acrylate copolymer can be a functionalized crosslinked acrylate random copolymer comprising randomly dispersed first units and second units.

Another embodiment is a curable composition comprising: an unfunctionalized uncrosslinked acrylate block copolymer comprising a first block and a second block; wherein the first block comprises first units derived from a first monomer selected from the group consisting of C₁₀-C₂₄-alkyl (meth)acrylates, C₁₀-C₂₄-alkenyl (meth)acrylates, C₁₀-C₂₄-alkyl (meth)acrylamides, C₁₀-C₂₄-alkenyl (meth)acrylamides, and combinations thereof; and the second block comprises second units is derived from a second monomer selected from the group consisting of (meth)acrylic acid, C₁-C₈-alkyl (meth)acrylates, C₁-C₈-alkyl (meth)acrylamides, 2-carboxyethyl (meth)acrylate, C₂-C₈-hydroxyalkyl (meth)acrylates, C₂-C₈-hydroxyalkyl (meth)acrylamides, ethylene glycol methyl ether (meth)acrylate, ethylene glycol methyl ether (meth)acrylamide, mono-2-(methacryloyloxy)ethyl maleate, mono-2-(methacryloyloxy)ethyl succinate, itaconic acid,

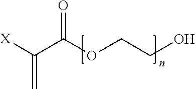

wherein X is hydrogen or methyl, and n is 2, 3, 4, 5, 6, 7, 8, 9, or 10, and combinations thereof; wherein the unfunctionalized uncrosslinked acrylate block copolymer comprises 15 to 65 mole percent of first units and 35 to 85 mole percent of second units, based on the total mole percent of first units and second units in the unfunctionalized uncrosslinked acrylate block copolymer; and a polymerizable monomer comprising a sixth monomer comprising two or more reactive groups selected from the group consisting of (meth)acryloyloxy groups, (meth)acrylamido groups, and combinations thereof.

This curable composition comprising an unfunctionalized uncrosslinked acrylate block copolymer and a polymerizable monomer is an effective alternative to the curable composition comprising a functionalized crosslinked acrylate copolymer and a copolymerizable monomer. Both approaches include formation of semicrystalline domains derived from the long hydrocarbon chain of the first monomer. When a functionalized crosslinked acrylate copolymer is used, the semicrystalline domains are confined, in part, by polymer interpenetration, chemical crosslinking involving the functional groups of the functionalized crosslinked acrylate copolymer, and physical crosslinks. When an unfunctionalized uncrosslinked acrylate block copolymer is used, the semicrystalline domains are confined, in part, by polymer interpenetration and physical crosslinks (as the unfunctionalized uncrosslinked acrylate block copolymer includes no polymerizable functional groups, it cannot contribute to chemical crosslinking, but chemical crosslinking is present due to the presence in the curable composition of the sixth monomer comprising two or more reactive groups).

The unfunctionalized uncrosslinked acrylate block copolymer comprises at least one first block and at least one second block. The first block comprises first units derived from a first monomer selected from the group consisting of C₁₀-C₂₄-alkyl (meth)acrylates, C₁₀-C₂₄-alkenyl (meth)acrylates, C₁₀-C₂₄-alkyl (meth)acrylamides, C₁₀-C₂₄-alkenyl (meth)acrylamides, and combinations thereof. The first monomer used to form the first block of the unfunctionalized uncrosslinked acrylate block copolymer is the same as the first monomer used to form the functionalized crosslinked acrylate copolymer described above. The second block comprises second units derived from a second monomer selected from the group consisting of (meth)acrylic acid, C₁-C₈-alkyl (meth)acrylates, C₁-C₈-alkyl (meth)acrylamides, 2-carboxyethyl (meth)acrylate, C₂-C₈-hydroxyalkyl (meth)acrylates, C₂-C₈-hydroxyalkyl (meth)acrylamides, ethylene glycol methyl ether (meth)acrylate, ethylene glycol methyl ether (meth)acrylamide, mono-2-(methacryloyloxy)ethyl maleate, mono-2-(methacryloyloxy)ethyl succinate, itaconic acid,

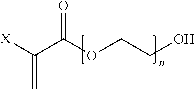

wherein X is hydrogen or methyl, and n is 2, 3, 4, 5, 6, 7, 8, 9, or 10, and combinations thereof. The second monomer used to form the second block of the unfunctionalized uncrosslinked acrylate block copolymer is the same as the second monomer used to form the functionalized crosslinked acrylate copolymer described above.

The unfunctionalized uncrosslinked acrylate block copolymer comprises 15 to 65 mole percent of first units and 35 to 85 mole percent of second units, based on the total mole percent of first units and second units in the unfunctionalized uncrosslinked acrylate block copolymer. Within the range of 15 to 65 mole percent, the content of first units in the unfunctionalized uncrosslinked acrylate block copolymer can be 25 to 65 mole percent, or 35 to 65 mole percent. Within the range of 35 to 85 mole percent, the content of second units in the unfunctionalized uncrosslinked acrylate block copolymer can be 35 to 75 mole percent, or 35 to 65 mole percent.

The unfunctionalized uncrosslinked acrylate block copolymer can be a diblock (AB) copolymer, a triblock (ABA, BAB) copolymer, a tetrablock (ABAB) copolymer, or a pentablock (ABABA, BABAB) copolymer. In some embodiments, the unfunctionalized uncrosslinked acrylate block copolymer is a diblock (AB) copolymer or a triblock (ABA, BAB) copolymer. In some embodiments, the unfunctionalized uncrosslinked acrylate block copolymer is a diblock (AB) copolymer.

The unfunctionalized uncrosslinked acrylate block copolymer can be synthesized by acrylate block copolymer preparation methods known in the art, including Reversible Addition Fragmentation Chain Transfer (RAFT). Synthesis of an unfunctionalized uncrosslinked acrylate diblock copolymer is described below in the working examples.

The curable composition comprising the unfunctionalized uncrosslinked acrylate block copolymer further comprises a polymerizable monomer. The polymerizable monomer comprises a sixth monomer comprising two or more reactive groups selected from the group consisting of (meth)acryloyloxy groups, (meth)acrylamido groups, and combinations thereof. In some embodiments, the sixth monomer comprises 2, 3, or 4 reactive groups selected from the group consisting of (meth)acryloyloxy groups, (meth)acrylamido groups, and combinations thereof. In some embodiments, the sixth monomer comprises 2 or 3 reactive groups selected from the group consisting of (meth)acryloyloxy groups, (meth)acrylamido groups, and combinations thereof. In some embodiments, the sixth monomer comprises 2 reactive groups selected from the group consisting of (meth)acryloyloxy groups, (meth)acrylamido groups, and combinations thereof.

The sixth monomer can be selected from, for example, diethyleneglycol di(meth)acrylate, triethyleneglycol di(meth)acrylate, tetraethyleneglycol di(meth)acrylate, bisphenol A glycerolate di(meth)acrylate, diurethane di(meth)acrylate, C₁-C₆-alkylene bis((meth)acrylate)s, C₁-C₆-alkylene bis((meth)acrylamide)s, trimethylolpropane tri(meth)acrylate, di(trimethylolpropane) tetra(meth)acrylate, polycaprolactone di(meth)acrylate, polycaprolactone tri(meth)acrylate, and combinations thereof.

In some embodiments, polymerizable monomer further comprises a seventh monomer comprising one (meth)acryloyloxy group or one (meth)acrylamido group. The seventh monomer can be selected from, for example, (meth)acrylic acid, C₁-C₈-alkyl (meth)acrylates, C₁-C₈-alkyl (meth)acrylamides, 2-carboxyethyl (meth)acrylate, C₂-C₈-hydroxyalkyl (meth)acrylates, C₂-C₈-hydroxyalkyl (meth)acrylamides, ethylene glycol methyl ether (meth)acrylate, ethylene glycol methyl ether (meth)acrylamide, mono-2-(methacryloyloxy)ethyl maleate, mono-2-(methacryloyloxy)ethyl succinate, itaconic acid,

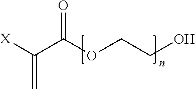

wherein X is hydrogen or methyl, and n is 2, 3, 4, 5, 6, 7, 8, 9, or 10, or a combination thereof.

In some embodiments, the curable composition comprises 2 to 25 weight percent of the unfunctionalized uncrosslinked acrylate copolymer, and 75 to 98 weight percent of the polymerizable monomer, based on the total weight of the curable composition. Within the range of 2 to 25 weight percent, the unfunctionalized uncrosslinked acrylate copolymer content of the curable composition can be 3 to 20 weight percent, or 5 to 15 weight percent. Within the range of 75 to 98 weight percent, the polymerizable monomer content of the curable composition can be 80 to 97 weight percent, or 85 to 95 weight percent.

Another embodiment is a cured composition comprising the product of curing a curable composition in any of its herein-described variations. Curing can be effected thermally, photochemically, or with a combination of thermal and photochemical methods. Catalysts for thermal and photochemical curing are known in the art.

The functionalized crosslinked acrylate copolymer and the unfunctionalized uncrosslinked acrylate copolymer have been described in terms of their use in curable and corresponding cured compositions. Such curable and cured compositions are useful as, for example acrylate-based adhesives in which the functionalized copolymer is an additive to the adhesive (including dental adhesives, adhesives for sign-making, adhesives for sporting goods, adhesives for automotive manufacturing, adhesives for aerospace manufacturing, adhesives for window-mount bonding and sealing, and adhesives for magnet bonding within electrical motors and speakers), acrylic resins in which the functionalized copolymer is chemically incorporated into the backbone of the resin (including filled dental resins, acrylic paints for artists, latex paints, and as one component of a two-component mixture of acrylic resin with pendant hydroxyl groups and an isocyanate crosslinker), vinyl ester resins in which the functionalized copolymer is an additive to the resin (including fiberglass-reinforced structural materials for marine and aerospace applications), and unsaturated polyester resins in which the functionalized copolymer is an additive to the resin (including glass fiber-reinforced composites, including those used for gel coats, shirt buttons, mine-bolts, bowling ball cores, polymer concrete, and engineered stone/cultured marble).

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

All cited patents, patent applications, and other references are incorporated herein by reference in their entirety. However, if a term in the present application contradicts or conflicts with a term in the incorporated reference, the term from the present application takes precedence over the conflicting term from the incorporated reference.

All ranges disclosed herein are inclusive of the endpoints, and the endpoints are independently combinable with each other. Each range disclosed herein constitutes a disclosure of any point or sub-range lying within the disclosed range.

The use of the terms “a” and “an” and “the” and similar referents in the context of describing the invention (especially in the context of the following claims) are to be construed to cover both the singular and the plural, unless otherwise indicated herein or clearly contradicted by context. Further, it should further be noted that the terms “first,” “second,” and the like herein do not denote any order, quantity, or importance, but rather are used to distinguish one element from another. The modifier “about” used in connection with a quantity is inclusive of the stated value and has the meaning dictated by the context (e.g., it includes the degree of error associated with measurement of the particular quantity).

The invention includes at least the following aspects.

Aspect 1: A functionalized crosslinked acrylate copolymer comprising: 15 to 63 mole percent of first units derived from a first monomer selected from the group consisting of C₁₀-C₂₄-alkyl (meth)acrylates, C₁₀-C₂₄-alkenyl (meth)acrylates, C₁₀-C₂₄-alkyl (meth)acrylamides, C₁₀-C₂₄-alkenyl (meth)acrylamides, and combinations thereof; 35 to 83 mole percent of second units derived from a second monomer selected from the group consisting of (meth)acrylic acid, C₁-C₈-alkyl (meth)acrylates, C₁-C₈-alkyl (meth)acrylamides, 2-carboxyethyl (meth)acrylate, C₂-C₈-hydroxyalkyl (meth)acrylates, C₂-C₈-hydroxyalkyl (meth)acrylamides, ethylene glycol methyl ether (meth)acrylate, ethylene glycol methyl ether (meth)acrylamide, mono-2-(methacryloyloxy)ethyl maleate, mono-2-(methacryloyloxy)ethyl succinate, itaconic acid,

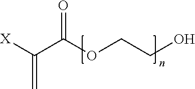

wherein X is hydrogen or methyl, and n is 2, 3, 4, 5, 6, 7, 8, 9, or 10, and combinations thereof; and 2 to 50 mole percent of third units derived from a third monomer selected from the group consisting of C₁-C₆-alkylene bis((meth)acrylate)s, C₁-C₆-alkylene bis((meth)acrylamide)s, ethylene glycol di(meth)acrylate, diethylene glycol di(meth)acrylate, triethylene glycol di(meth)acrylate, tetraethylene glycol di(meth)acrylate, bisphenol A glycerolate di(meth)acrylate, diurethane di(meth)acrylate, trimethylolpropane tri(meth)acrylate, di(trimethylolpropane) tetra(meth)acrylate, polycaprolactone di(meth)acrylate, polycaprolactone tri(meth)acrylate, and combinations thereof; wherein the functionalized crosslinked acrylate copolymer comprises 0.6 to 25 mole percent of reactive functional groups selected from the group consisting of (meth)acrylate, (meth)acrylamido, and combinations thereof; and wherein all mole percent values are based on total moles of first units, second units, and third units in the functionalized crosslinked acrylate copolymer.

Aspect 2: The functionalized crosslinked acrylate copolymer of aspect 1, wherein the first monomer is selected from the group consisting of C₁₂-C₂₂-alkyl (meth)acrylates, and combinations thereof.

Aspect 3: The functionalized crosslinked acrylate copolymer of aspect 1 or 2, wherein the second monomer is selected from the group consisting of C₁-C₈-alkyl (meth)acrylates, C₁-C₈-alkyl (meth)acrylamides, 2-carboxyethyl (meth)acrylate, C₂-C₈-hydroxyalkyl (meth)acrylates, C₂-C₈-hydroxyalkyl (meth)acrylamides, ethylene glycol methyl ether (meth)acrylate, ethylene glycol methyl ether (meth)acrylamide, mono-2-(methacryloyloxy)ethyl maleate, mono-2-(methacryloyloxy)ethyl succinate, itaconic acid,

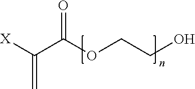

wherein X is hydrogen or methyl, and n is 2, 3, 4, 5, 6, 7, 8, 9, or 10, and combinations thereof.

Aspect 4: The functionalized crosslinked acrylate copolymer of aspect 1 or 2, wherein the second monomer is selected from the group consisting of C₁-C₈-alkyl (meth)acrylates, C₁-C₈-alkyl (meth)acrylamides, 2-carboxyethyl (meth)acrylate, C₂-C₈-hydroxyalkyl (meth)acrylates, C₂-C₈-hydroxyalkyl (meth)acrylamides, ethylene glycol methyl ether (meth)acrylate, ethylene glycol methyl ether (meth)acrylamide, mono-2-(methacryloyloxy)ethyl maleate, mono-2-(methacryloyloxy)ethyl succinate, itaconic acid,

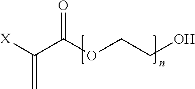

wherein X is hydrogen or methyl, and n is 2, 3, 4, 5, 6, 7, 8, 9, or 10, and combinations thereof, provided that if the second monomer comprises a C₁-C₈-alkyl (meth)acrylamide, it excludes N,N-dimethylacrylamide.

Aspect 5: The functionalized crosslinked acrylate copolymer of aspect 1 or 2, wherein the second monomer is selected from the group consisting of acrylic acid, ethyl acrylate, butyl acrylate, 2-ethylhexyl acrylate, 2-carboxyethyl acrylate, ethylene glycol methyl ether acrylate, N,N-dimethyl acrylamide, N-iso-propyl acrylamide, N-tert-butyl acrylamide, N-hydroxyethyl acrylamide, 2-hydroxypropyl methacrylamide, N-[tris(hydroxymethyl)methyl]acrylamide,

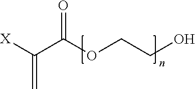

wherein X is hydrogen or methyl, and n is 2, 3, 4, 5, 6, 7, 8, 9, or 10, and combinations thereof.

Aspect 6: The functionalized crosslinked acrylate copolymer of any one of aspects 1-5, wherein the third monomer is selected from the group consisting of N,N′-methylenebis((meth)acrylamide), 1,4-butanediol di(meth)acrylate, 1,6-hexanediol di(meth)acrylate, ethylene glycol di(meth)acrylate, diethylene glycol di(meth)acrylate, triethylene glycol di(meth)acrylate, and combinations thereof.

Aspect 7: The functionalized crosslinked acrylate copolymer of any one of aspects 1-6, wherein the functionalized crosslinked acrylate copolymer is a functionalized crosslinked acrylate random copolymer comprising randomly dispersed first units and second units.

Aspect 8: The functionalized crosslinked acrylate copolymer of any one of aspects 1-7, further comprising 0.5 to 5 mole percent of hydroxy-functionalized azobenzene groups.

Aspect 9: The functionalized crosslinked acrylate copolymer of aspect 1, comprising: 15 to 60 mole percent of the first units; wherein the first monomer is selected from the group consisting of C₁₂-C₂₂-alkyl (meth)acrylates, and combinations of C₁₂-C₂₂-alkyl (meth)acrylates; 15 to 60 mole percent of the second units; wherein the second monomer is selected from the group consisting of acrylic acid, ethyl acrylate, butyl acrylate, 2-ethylhexyl acrylate, 2-carboxyethyl acrylate, ethylene glycol methyl ether acrylate, N,N-dimethyl acrylamide, N-iso-propyl acrylamide, N-tert-butyl acrylamide, N-hydroxyethyl acrylamide, 2-hydroxypropyl methacrylamide, N-[tris(hydroxymethyl)methyl]acrylamide, and combinations thereof; and 15 to 35 mole percent of the third units; wherein the third monomer is selected from the group consisting of N,N′-methylenebis((meth)acrylamide), ethylene glycol di(meth)acrylate, 1,4-butanediol di(meth)acrylate, 1,6-hexanediol di(meth)acrylate, diethylene glycol di(meth)acrylate, triethylene glycol di(meth)acrylate, and combinations thereof; wherein the functionalized crosslinked acrylate copolymer comprises 2 to 20 mole percent of the reactive functional groups.

Aspect 10: The functionalized crosslinked acrylate copolymer of aspect 1, comprising: 15 to 60 mole percent of the first units; wherein the first monomer is selected from the group consisting of C₁₂-C₂₂-alkyl (meth)acrylates, and combinations of C₁₂-C₂₂-alkyl (meth)acrylates; 15 to 60 mole percent of the second units; wherein the second monomer is selected from the group consisting of acrylic acid, 2-carboxyethyl acrylate, N,N-dimethyl acrylamide, N-iso-propyl acrylamide, N-tert-butyl acrylamide, N-hydroxyethyl acrylamide, 2-hydroxypropyl methacrylamide, N-[tris(hydroxymethyl)methyl]acrylamide, and combinations thereof; and 15 to 35 mole percent of the third units; wherein the third monomer is selected from the group consisting of N,N′-methylenebis((meth)acrylamide), ethylene glycol di(meth)acrylate, 1,4-butanediol di(meth)acrylate, 1,6-hexanediol di(meth)acrylate, diethylene glycol di(meth)acrylate, triethylene glycol di(meth)acrylate, and combinations thereof; wherein the functionalized crosslinked acrylate copolymer comprises 2 to 20 mole percent of the reactive functional groups.

Aspect 11: A method of forming a functionalized crosslinked acrylate copolymer, comprising: partially copolymerizing a monomer mixture comprising a first monomer selected from the group consisting of C₁₀-C₂₄-alkyl (meth)acrylates, C₁₀-C₂₄-alkenyl (meth)acrylates, C₁₀-C₂₄-alkyl (meth)acrylamides, C₁₀-C₂₄-alkenyl (meth)acrylamides, and combinations thereof; a second monomer selected from the group consisting of (meth)acrylic acid, C₁-C₈-alkyl (meth)acrylates, C₁-C₈-alkyl (meth)acrylamides, 2-carboxyethyl (meth)acrylate, C₂-C₈-hydroxyalkyl (meth)acrylates, C₂-C₈-hydroxyalkyl (meth)acrylamides, ethylene glycol methyl ether (meth)acrylate, ethylene glycol methyl ether (meth)acrylamide, mono-2-(methacryloyloxy)ethyl maleate, mono-2-(methacryloyloxy)ethyl succinate, itaconic acid,

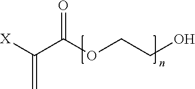

wherein X is hydrogen or methyl, and n is 2, 3, 4, 5, 6, 7, 8, 9, or 10, and combinations thereof; and a third monomer selected from the group consisting of C₁-C₆-alkylene bis((meth)acrylate)s, C₁-C₆-alkylene bis((meth)acrylamide)s, diethylene glycol di(meth)acrylate, triethylene glycol di(meth)acrylate, tetraethylene glycol di(meth)acrylate, bisphenol A diglycidyl ether di(meth)acrylate, diurethane di(meth)acrylate, trimethylolpropane tri(meth)acrylate, di(trimethylolpropane) tetra(meth)acrylate, polycaprolactone di(meth)acrylate, polycaprolactone tri(meth)acrylate, and combinations thereof to form a functionalized crosslinked acrylate copolymer comprising 15 to 63 mole percent of first units derived from the first monomer, 35 to 83 mole percent of second units derived from the second monomer, and 2 to 50 mole percent of third units derived from the third monomer; wherein of the 2 to 50 mole percent of third units derived from the third monomer, 0.6 to 25 mole percent have at least one pendant (meth)acryloyloxy or (meth)acrylamido group, and 25 to 49.4 mole percent have zero pendant (meth)acryloyloxy or (meth)acrylamido groups; and wherein all mole percent values are based on total moles of first units, second units, and third units in the functionalized crosslinked acrylate copolymer.

Aspect 12: The method of aspect 11, wherein the partially copolymerizing comprises polymerizing 50 to 80 mole percent of polymerizable groups contributed by the first monomer, second monomer, and third monomer.

Aspect 13: The method of aspect 11 or 12, wherein the second monomer comprises the C₂-C₈-hydroxyalkyl (meth)acrylate, the C₂-C₈-hydroxyalkyl (meth)acrylamide, or a combination thereof, and at least a portion of the second units comprise pendant hydroxyl groups; and wherein the method further comprises reacting at least a portion of the pendant hydroxyl groups with a reagent capable of producing a pendant (meth)acrylate group.

Aspect 14: The method of any one of aspects 11 to 13, wherein the functionalized crosslinked acrylate copolymer comprises: 20 to 50 mole percent of the first units; wherein the first monomer is selected from the group consisting of C₁₂-C₂₂-alkyl (meth)acrylates, and combinations of C₁₂-C₂₂-alkyl (meth)acrylates; 45 to 75 mole percent of the second units; wherein the second monomer is selected from the group consisting of acrylic acid, ethyl acrylate, butyl acrylate, 2-ethylhexyl acrylate, 2-carboxyethyl acrylate, ethylene glycol methyl ether acrylate, N,N-dimethyl acrylamide, N-iso-propyl acrylamide, N-tert-butyl acrylamide, N-hydroxyethyl acrylamide, 2-hydroxypropyl methacrylamide, N-[tris(hydroxymethyl)methyl]acrylamide,

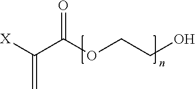

wherein X is hydrogen or methyl, and n is 2, 3, 4, 5, 6, 7, 8, 9, or 10, and combinations thereof; and 5 to 35 mole percent of the third units; wherein the third monomer is selected from the group consisting of N,N′-methylenebis((meth)acrylamide), ethylene glycol di(meth)acrylate, 1,4-butanediol di(meth)acrylate, 1,6-hexanediol di(meth)acrylate, diethylene glycol di(meth)acrylate, triethylene glycol di(meth)acrylate, and combinations thereof; wherein the functionalized crosslinked acrylate copolymer comprises 2 to 20 mole percent of the reactive functional groups.

Aspect 15: A curable composition comprising: the functionalized crosslinked acrylate copolymer of any one of aspects 1-11; and a copolymerizable monomer.

Aspect 16: The curable composition of aspect 15, comprising, based on the total weight of the curable composition, 2 to 25 weight percent of the functionalized crosslinked acrylate copolymer; and 75 to 98 weight percent of the copolymerizable monomer; wherein the copolymerizable monomer comprises, based on 100 parts by weight of the copolymerizable monomer, 80 to 95 parts by weight of a fourth monomer selected from the group consisting of (meth)acrylic acid, C₁-C₈-alkyl (meth)acrylates, C₁-C₈-alkyl (meth)acrylamides, 2-carboxyethyl (meth)acrylate, C₂-C₈-hydroxyalkyl (meth)acrylates, C₂-C₈-hydroxyalkyl (meth)acrylamides, ethylene glycol methyl ether (meth)acrylate, ethylene glycol methyl ether (meth)acrylamide, mono-2-(methacryloyloxy)ethyl maleate, mono-2-(methacryloyloxy)ethyl succinate, itaconic acid, and combinations thereof; and 5 to 20 parts by weight of a fifth monomer selected from the group consisting of diethyleneglycol di(meth)acrylate, triethyleneglycol di(meth)acrylate, tetraethyleneglycol di(meth)acrylate, bisphenol A glycerolate di(meth)acrylate, diurethane di(meth)acrylate, C₁-C₆-alkylene bis((meth)acrylate)s, C₁-C₆-alkylene bis((meth)acrylamide)s, trimethylolpropane tri(meth)acrylate, di(trimethylolpropane) tetra(meth)acrylate, polycaprolactone di(meth)acrylate, polycaprolactone tri(meth)acrylate, and combinations thereof.

Aspect 17: The curable composition of aspect 15 or 16, wherein the functionalized crosslinked acrylate copolymer comprises: 15 to 60 mole percent of the first units; wherein the first monomer is selected from the group consisting of C₁₂-C₂₂-alkyl (meth)acrylates, and combinations of C₁₂-C₂₂-alkyl (meth)acrylates; 15 to 60 mole percent of the second units; wherein the second monomer is selected from the group consisting of acrylic acid, ethyl acrylate, butyl acrylate, 2-ethylhexyl acrylate, 2-carboxyethyl acrylate, ethylene glycol methyl ether acrylate, N,N-dimethyl acrylamide, N-iso-propyl acrylamide, N-tert-butyl acrylamide, N-hydroxyethyl acrylamide, 2-hydroxypropyl methacrylamide, N-[tris(hydroxymethyl)methyl]acrylamide,

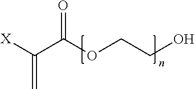

wherein X is hydrogen or methyl, and n is 2, 3, 4, 5, 6, 7, 8, 9, or 10, and combinations thereof; 15 to 35 mole percent of the third units; wherein the third monomer is selected from the group consisting of N,N′-methylenebis((meth)acrylamide), ethylene glycol di(meth)acrylate, 1,4-butanediol di(meth)acrylate, 1,6-hexanediol di(meth)acrylate, diethylene glycol di(meth)acrylate, triethylene glycol di(meth)acrylate, and combinations thereof; wherein the functionalized crosslinked acrylate copolymer comprises 2 to 20 mole percent of the reactive functional groups.

Aspect 18: The curable composition of any one of aspects 15-17, wherein the functionalized crosslinked acrylate copolymer comprises a first functionalized crosslinked acrylate copolymer comprising second units derived from a second monomer selected from the group consisting of C₂-C₈-hydroxyalkyl (meth)acrylates, C₂-C₈-hydroxyalkyl (meth)acrylamides,

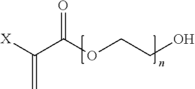

wherein X is hydrogen or methyl, and n is 2, 3, 4, 5, 6, 7, 8, 9, or 10, and combinations thereof; and a second functionalized crosslinked acrylate copolymer comprising second units derived from a second monomer selected from the group consisting of (meth)acrylic acid, mono-2-(methacryloyloxy)ethyl maleate, mono-2-(methacryloyloxy)ethyl succinate, itaconic acid, and combinations thereof; wherein the curable composition comprises, based on the total weight of the curable composition, 2 to 15 weight percent of the first functionalized crosslinked acrylate copolymer and 2 to 15 weight percent of the second functionalized crosslinked acrylate copolymer.

Aspect 19: A cured composition comprising the product of curing the curable composition of any one of aspects 15 to 18.

Aspect 20: A curable composition comprising: an unfunctionalized uncrosslinked acrylate block copolymer comprising a first block and a second block; wherein the first block comprises first units derived from a first monomer selected from the group consisting of C₁₀-C₂₄-alkyl (meth)acrylates, C₁₀-C₂₄-alkenyl (meth)acrylates, C₁₀-C₂₄-alkyl (meth)acrylamides, C₁₀-C₂₄-alkenyl (meth)acrylamides, and combinations thereof; and the second block comprises second units is derived from a second monomer selected from the group consisting of (meth)acrylic acid, C₁-C₈-alkyl (meth)acrylates, C₁-C₈-alkyl (meth)acrylamides, 2-carboxyethyl (meth)acrylate, C₂-C₈-hydroxyalkyl (meth)acrylates, C₂-C₈-hydroxyalkyl (meth)acrylamides, ethylene glycol methyl ether (meth)acrylate, ethylene glycol methyl ether (meth)acrylamide, mono-2-(methacryloyloxy)ethyl maleate, mono-2-(methacryloyloxy)ethyl succinate, itaconic acid,

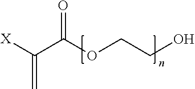

wherein X is hydrogen or methyl, and n is 2, 3, 4, 5, 6, 7, 8, 9, or 10, and combinations thereof; wherein the unfunctionalized uncrosslinked acrylate block copolymer comprises 15 to 65 mole percent of first units and 35 to 85 mole percent of second units, based on the total mole percent of first units and second units in the unfunctionalized uncrosslinked acrylate block copolymer; and a polymerizable monomer comprising a sixth monomer comprising two or more reactive groups selected from the group consisting of (meth)acryloyloxy groups, (meth)acrylamido groups, and combinations thereof.

Aspect 21: The curable composition of aspect 20, wherein the polymerizable monomer further comprises a seventh monomer comprising one (meth)acryloyloxy group or one (meth)acrylamido group.

Aspect 22: A cured composition comprising the product of curing the curable composition of claim 20 or 21.

The invention is further illustrated by the following non-limiting examples.

### Examples

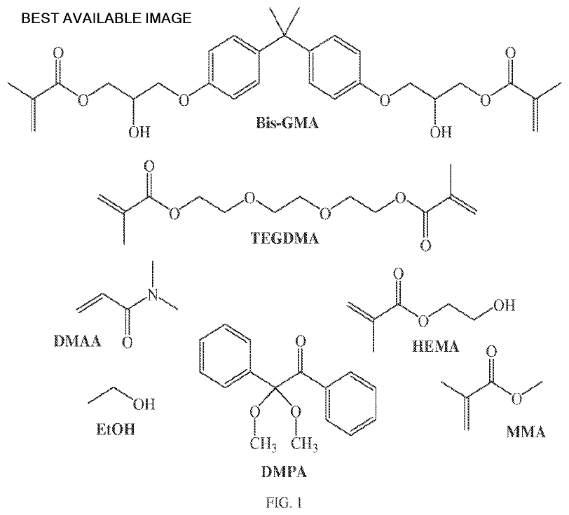

Table 1 summarizes materials used in these examples. Chemical structures of some monomers, solvents, and initiators are provided in FIG. 1. All materials were obtained from Sigma.

TABLE 1

| Material | Description |
|---|---|
| SA | stearyl acrylate, CAS Reg. No. 4813-57-4 |
| EHA | 2-ethylhexyl acrylate, CAS Reg. No. 103-11-7 |
| AA | acrylic acid, CAS Reg. No. 79-10-7 |
| DMAA | N,N-dimethylacrylamide, CAS Reg. No. 2680-03-7 |
| MBAA | N,N′-methylenebis(acrylamide), CAS Reg. No. 110-26-9 |
| MMA | methyl methacrylate, CAS Reg. No. 80-62-6 |
| HEMA | 2-hydroxyethyl methacrylate, CAS Reg. No. 868-77-9 |
| MMES | mono-2-(methacryloyloxy)ethyl succinate, CAS Reg. No. |
| AIBN | 2,2′-azobis(isobutyronitrile), CAS Reg. No. 78-67-1 |
| EtOH | ethanol, CAS Reg. No. 64-17-5 |
| BisGMA | bisphenol A glycerolate dimethacrylate, CAS Reg. No. 1565-94-2 |
| PMMA | poly(methyl methacrylate), CAS Reg. No. 9011-14-7, having a |
| | weight average molecular weight of 120,000 grams/mole |
| TEGDMA | tetraethylene glycol dimethacrylate, CAS Reg. No. 109-17-1 |
| DBTTC | S-dibenzyl trithiocarbonate, CAS Reg. No. 26504-29-0 |
| DMPA | 2,2-dimethoxy-2-phenylacetophenone, CAS Reg. No. 24650-42-8 |

Both random and block copolymers were synthesized with stearyl acrylate (SA) at 22.5-50 mole percent, in combination with 2-ethylhexyl acrylate (EHA) or acrylic acid (AA) or N,N-dimethylacrylamide (DMAA) or 2-hydroxyethyl methacrylate (HEMA) at 47.5-75 mole percent, and the crosslinker N,N′-methylenebisacrylamide (MBAA) at 0-5 mole percent, wherein all mole percent values are based on total moles of SA, EHA, AA, DMAA, HEMA, and MBAA. SA, with an eighteen carbon alkyl group, was chosen because side-chains with at least twelve carbon atoms tend to form crystalline structures with long-range order. See, e.g., S. A. Greenberg and T. Alfrey, “Side Chain Crystallization of n-Alkyl Polymethacrylates and Polyacrylates,” Journal of the American Chemical Society 1954, volume 76, pages 6280-6285. In addition to imparting the requisite balance of hydrophobicity and hydrophilicity, and tunable thermomechanical properties, acrylate and acrylamide monomers in the prepolymerized copolymer format (i.e., the copolymer obtained by partial polymerization of the reaction mixture) were chosen for their reactivity (i.e., residual acrylamide groups derived from MBAA) and biocompatibility. The monomers for the crosslinked networks (i.e., the copolymerizable monomers) were chosen based on the miscibility of the synthesized functionalized copolymer and were a combination of hydrophilic (TEGDMA, DMAA, HEMA) and hydrophobic (EHA and MMA) monomers.

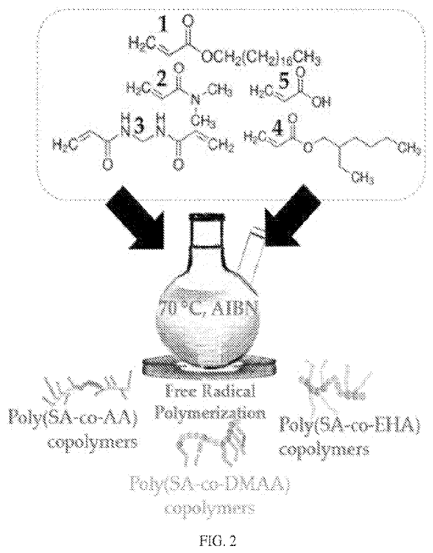

Functionalized crosslinked random copolymers designated poly(SA-co-EHA), poly(SA-co-AA), poly(SA-co-DMAA), and poly(SA-co-HEMA) were synthesized using a modification of a previously published procedure for the preparation of unfunctionalized crosslinked random copolymers of stearyl acrylate and acrylic acid. A. Matsuda, J. Sato, H. Yasunaga, and Y. Osada, “Order-Disorder Transition of a Hydrogel Containing an n-Alkyl Acrylate,” Macromolecules 1994, volume 27, pages 7695-7698. Briefly, copolymers with various molar ratios of SA and MBAA and either EHA, DMAA, or AA were prepared by free-radical copolymerization as illustrated in FIG. 2, in which the monomers were dispersed in ethanol and the reaction was initiated via azobisisobutyronitrile (AIBN) at 70° C. and monitored via Fourier transform infrared spectroscopy (FTIR; mid-IR acrylic C═C peaks at 814 cm⁻¹). The reaction was stopped at 70% (meth)acrylate/(meth)acrylamide double bond conversion to ensure residual functionality within the copolymers for subsequent polymerization. The crude reaction mixture was immersed in excess ethanol to remove unreacted monomers, uncrosslinked polymers, and initiator.

In Table 2, the random copolymer designated “Poly(SA-co-AA2575)” was a random copolymer prepared with a molar ratio of SA to HEMA to MBAA of 22.5:72.5:5, using four volumes ethanol as a solvent and about 70 mole percent conversion of (meth)acrylate/(meth)acrylamide groups based on monitoring by FTIR at 815 centimeter⁻¹. The random copolymer designated “Poly(SA-co-EHA5050)” was prepared with a molar ratio of SA to EHA to MBAA of 47.5:47.5:5, using four volumes ethanol as a solvent and about 70 mole percent conversion of (meth)acrylate/(meth)acrylamide groups based on monitoring by FTIR at 815 centimeter⁻¹. The random copolymer designated “Poly(SA-co-EHA2575)” was prepared with a molar ratio of SA to EHA to MBAA of 25:74:1, using four volumes ethanol as a solvent and about 70 mole percent conversion of (meth)acrylate/(meth)acrylamide groups based on monitoring by FTIR at 815 centimeter⁻¹. The random copolymer designated “Poly(SA-co-DMAA2575)” was prepared with a molar ratio of SA to DMAA to MBAA of 22.5:72.5:5, using four volumes ethanol as a solvent and about 70 mole percent conversion of (meth)acrylate/(meth)acrylamide groups based on monitoring by FTIR at 815 centimeter⁻¹. The random copolymer designated “Poly(SA-co-DMAA5050)” was prepared with a molar ratio of SA to DMAA to MBAA of 47.5:47.5:5, using four volumes ethanol as a solvent and about 70 mole percent conversion of (meth)acrylate/(meth)acrylamide groups based on monitoring by FTIR at 815 centimeter⁻¹. The random copolymer designated “Poly(SA-co-HEMA5050)” was prepared with a molar ratio of SA to HEMA to MBAA of 47.5:47.5:5, using four volumes ethanol as a solvent and about 70 mole percent conversion of (meth)acrylate/(meth)acrylamide groups based on monitoring by FTIR at 815 centimeter⁻¹.

A miniemulsion protocol for an unfunctionalized uncrosslinked ABA triblock copolymer was adapted to synthesize an unfunctionalized uncrosslinked AB diblock copolymer with SA and EHA blocks using Reversible Addition Fragmentation Chain Transfer (RAFT). G. Siljanovska Petreska, C. Auschra, and M. Paulis, “Confinement driven crystallization of ABA crystalline-soft-crystalline block copolymers synthesized via RAFT mediated miniemulsion polymerization,” Polymer 2018, volume 158, pages 327-337. Briefly, the synthesis of the SA block (Block A) was carried out by preparing an aqueous phase by dissolving the surfactant sodium dodecyl sulfate (SDS) in deionized water. Subsequently, AIBN and DBTTC were added to the preheated aqueous phase and the reaction was initiated at 70° C. and the polymerization proceeded under nitrogen. Once the formation of Block A was confirmed via proton nuclear magnetic resonance spectroscopy (¹H NMR) and gel permeation chromatography (GPC), a similar process was followed for the synthesis of Block B, with EHA. The molar ratio of (RAFT):(Initiator)=2:1 was maintained, and at the end of the reaction, the latex was filtered and collected. The mole ratio of SA units to EHA units was 1:1.

Number average molecular weight (Mₙ), weight average molecular weight (M_{w}), and polydispersity index (PDI=M_{w}/Mₙ) were determined for random and block copolymers by triple detection GPC using a series of four columns spanning molecular weights of 10⁴ to 10⁷ grams/mole, tetrahydrofuran eluent, a flow rate of 1 milliliter/minute, poly(methyl methacrylate) molecular weight standards, and refractive index and viscosity and light scattering detection. The light scattering signal was used to calculate Mₙ, M_{w}, and PDI. ¹H NMR analysis of the unfunctionalized uncrosslinked diblock copolymer confirmed the presence of distinct A (poly(stearyl acrylate)) and B (poly(2-ethylhexyl acrylate)) blocks.

The crosslinked copolymers were further characterized by Differential Scanning Calorimetry (DSC) and Dynamic Mechanical Analysis (DMA) to study the thermal and water-induced transitions of copolymers to establish the self-assembly in the networks. The mechanical changes as a function of temperature transitions measured in the DMA are more dramatic than changes in heat capacity (DSC), and as mechanical behavior is more pertinent to this study, DMA rather than DSC was used to determine the glass transition temperature (T_{g}) and melting temperature (Tₘ) values in Table 2. The bulk characteristics of the functionalized crosslinked copolymers were ascertained using the pockets method in the DMA. Anonymous, “Use of Material Pockets for Mechanical Analysis of Powders,” available at https://www.perkinelmer.com/lab-solutions/resources/docs/APP_007771B_03_Use_of_Material_Pockets_for_Mechanical_Analysi s_of_Powders.pdf. Briefly, a stainless steel envelope with the sample is mounted in the DMA instrument and then analyzed. Stainless steel does not have relaxations or transitions over the temperature range of the instrument and is an ideal sample mounting material. Dry and wet (24 hours in water at 37° C.) samples were measured with temperature sweeps from −80° C. to 100° C.

Properties of the functionalized crosslinked copolymers are summarized in Table 2.

TABLE 2

| | Weight average | | | Melting |
|---|---|---|---|---|
| | molecular | PDI | Tan Delta | Temperature |
| Copolymer designation | weight (M_{w}) | (M_{w}/Mₙ) | Peak (T_{g}) | (Tₘ) |
| Poly(SA-co-AA2575) | 22 kDa/mol | 2.36 | −5° C.*, | 33° C. |
| | | | 21° C. | |
| Poly(SA-co-EHA5050) | 462 kDa/mol | 1.38 | −30° C. | 45° C. |
| Poly(SA-co-EHA2575) | 318 kDa/mol | 1.57 | −28° C. | 50° C. |
| Poly(SA-co-DMAA2575) | 245 kDa/mol | 1.11 | 45° C. | >100° C. |
| Poly(SA-co-DMAA5050) | 388 kDa/mol | 1.07 | 40.5° C. | >100° C. |
| Poly(SA-co-HEMA5050) | 390 kDa/mol | 1.73 | −2.5° C.* | >100° C. |
| Poly(SA-b-EHA)** | 9.5 kDa/mol | 1.71 | −40° C. | 50° C. |

*measured under wet conditions
**diblock copolymer

Flexural properties were determined at 25° C. according to a modified ASTM D790 protocol on an MTS Mini Bionix™ II hydraulic universal testing machine.

The functionalized crosslinked copolymers were further characterized by Differential Scanning Calorimetry (DSC) and Dynamic Mechanical Analysis (DMA) to study the thermal and water-induced transitions of copolymers to establish the self-assembly in the networks. The mechanical changes as a function of temperature transitions measured in the DMA are more dramatic than changes in heat capacity (DSC), and as mechanical behavior is more pertinent to this study, DMA rather than DSC curves was used to characterize the functionalized crosslinked copolymers and functionalized crosslinked copolymer-loaded polymer substrates.

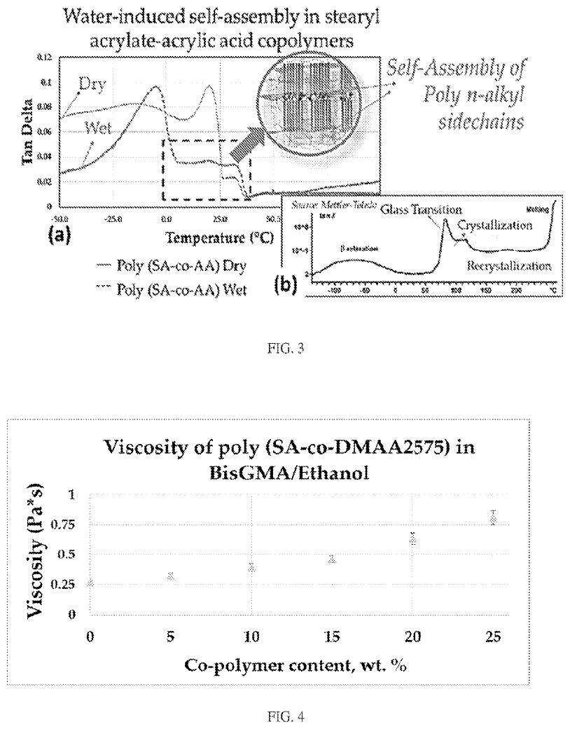

The bulk characteristics of the functionalized crosslinked copolymers were ascertained using the pockets method in the DMA. Anonymous, “Use of Material Pockets for Mechanical Analysis of Powders,” available at https://www.perkinelmer.com/lab-solutions/resources/docs/APP_007771B_03_Use_of_Material_Pockets_for_Mechanical_Analysi s_of_Powders.pdf. Briefly, a stainless steel envelope with the sample is mounted in the DMA instrument and then analyzed. Stainless steel does not have relaxations or transitions over the temperature range of the instrument and is an ideal sample mounting material. Dry and wet (24 hours in water at 37° C.) samples were measured with temperature sweeps from −80° C. to 100° C. The glass transition temperature (T_{g}) and melting temperature (Tₘ) of the functionalized crosslinked copolymers for dry samples are provided in Table 2. A representative graph for poly(SA-co-AA2575) measured both wet and dry is shown in FIG. 3(a). The cytocompatibility of the functionalized crosslinked copolymer was evaluated using an elution assay following a 24 hour incubation period. L. T. Poskus, R. S. M. S. Lima, I. Lima, J. G. Guimaraes, E. M. da Silva, and J. Granjeiro, “Cytotoxicity of current adhesive systems: in vitro testing on cell culture of L929 and balb/c 3T3 fibroblasts,” Revista Odonto Ciência 2009, volume 24, pages 129-134. Briefly, a series of dilutions of the extracted media at 25, 12.5, 6.25, and 0 weight percent was added to L929 cells and incubated for an additional 24 hours after which 3-(4,5-dimethylthiazol-2-yl)-2,5-diphenyl tetrazolium bromide (MTT reagent) was added. All concentrations tested were deemed cytocompatible. FIG. 3(b) is an idealized image from Mettler Toledo (not data generated by the present inventors) showing that dynamic mechanical analysis can detect transitions associated with crystallization of side chains from the first monomer. Crystallinity is typically dependent on the thermal history of the material. A sample can show crystallization upon heating. For a sample that has previously been cooled very quickly and has had no time to crystallize, heating above the glass transition yields small crystallites formed at relatively low temperatures. This is cold crystallization. On further heating, larger crystallites are formed from smaller crystallites. This is the recrystallization process.

FIG. 4 is a plot of viscosity (in units of millipascal-seconds) as a function of weight percent of the functionalized copolymer poly(SA-co-DMAA2575) in a solvent consisting of a 2:1 weight ratio of BisGMA to ethanol. The reactive polymer was the polymerization product of stearyl acrylate (SA) and N,N-dimethylacrylamide (DMAA) in a 25:75 mole ratio. BisGMA is propane-2,2-diylbis[4,1-phenyleneoxy(2-hydroxypropane-3,1-diyl)]bis(2-methylprop-2-enoate), CAS Reg. No. 1565-94-2. To determine viscosities, 15 milliliters of each mixture was placed in a Viscometer (CAP2000+; Brookfield, Middleboro, MA, USA). After a hold time of 5 seconds, the machine estimated the viscosity of the sample over a 10 second run at 25° C. Low viscosity is critical for a dental adhesive, which must flow easily across the tooth surface, infiltrate interfibrillar spaces and achieve high double-bond acrylic conversions upon photopolymerization (high double-bond conversion contributes to high strength). The reactive polymer's degree of branching, weight average molecular weight, and glass transition temperature, as well as ethanol content will determine viscosity and therefore the limit of RP within parent networks. While there is no defined optimum viscosity for adhesives, studies have shown that maintaining the viscosity of the monomers≤30 millipascal-seconds efficiently coats the tooth and results in ≥70% double-bond conversion upon photopolymerization.

Functionalized crosslinked copolymers that can solvate within the monomer formulations and form crosslinked networks were photopolymerized and studied. As shown in FIG. 5(a), up to 15 weight percent addition of the functionalized crosslinked random copolymer poly(SA-co-DMAA2575) within a 10/90 TEGDMA/DMAA formulation photopolymerized with high double-bond conversion within 60 seconds of light exposure. Samples were cured via exposure to 320-500 nanometer light at 45 milliwatts/centimeter² with the photoinitiator DMPA at 1 weight percent. Photopolymerizing functionalized crosslinked copolymer dispersed within monomers confines the long side-chains abruptly within narrow volumes with limited mobility and this drives the formation of self-assembled ordered morphologies within networks.

Thermomechanical analysis can quantify the ability of functionalized crosslinked copolymer to form self-assembling structures within substrates by registering the contribution from the presence of side-groups that are ordered and undergo segmental motion. A. Shrivastava, Introduction to Plastics Engineering 2018, pages 1-16, doi:10.1016/b978-0-323-39500-7.00001-0. FIG. 5(b) shows the results of dynamic mechanical analysis of the sample. Significantly, the presence of the functionalized crosslinked copolymer was able to induce changes in viscoelastic properties and introduce a “leathery region” within the substrate well below the T_{g} of the substrate (and in a clinically relevant temperature range), indicating the presence of side-groups and cooperative segmental motion within the sample. Functionalized crosslinked copolymers with T_{g} (45° C., Table 2) in the glassy, brittle region of the tan delta curve can impart mobility and enhanced toughness within the glassy network while a Tₘ>100° C. contributes to preserving the high strength and modulus, the high double-bond conversion, and the polymerization kinetics of the parent network.

In the oral cavity, water will eventually equilibrate into materials, whether they are hydrophilic or hydrophobic in nature. See, e.g., M. Cadenaro et al. “The role of polymerization in adhesive dentistry,” Dental Materials 2019, volume 35, pages e1-e22. In adhesive networks with functionalized crosslinked copolymer, post-polymerization exposure to water is believed to result in further crystallization of side-chains to form additional ordered domains that act as barriers within the submicron length scale and delay and/or reduce the further entry of water under clinically relevant conditions. However, any consideration of the effects of water in the oral cavity is incomplete without examining the effect of water during the photopolymerization of resins.

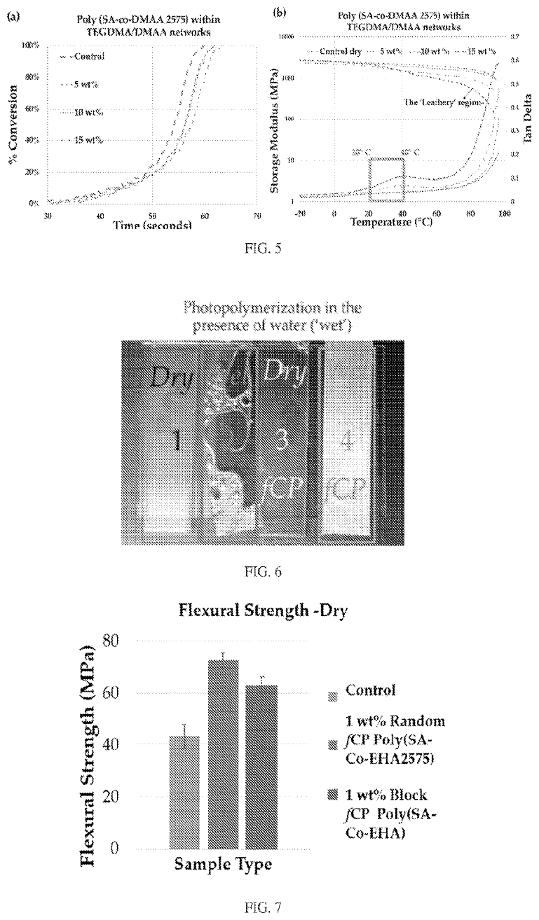

Despite the presence of ethanol, water present during photopolymerization can cause phase separation and is detrimental to the strength of the restoration. X. Guo, Y. Wang, P. Spencer, Q. Ye, and X. Yao, “Effects of water content and initiator composition on photopolymerization of a model BisGMA/HEMA resin,” Dental Materials, 2008, volume 24, pages 824-831. Additionally, the polymerization efficiency in each phase can differ dramatically because of the segregation of the initiator in addition to basic reactivity differences between the varied comonomer compositions. L. Song, R. Sarikaya, Q. Ye, A. Misra, C. Tamerler, and P. Spencer, “Multifunctional monomer acts as co-initiator and crosslinker to provide autonomous strengthening with enhanced hydrolytic stability in dental adhesives,” Dental Materials, 2020, volume 36, pages 284-295. We hypothesized that the incorporation of amphiphilic functionalized crosslinked copolymer within a mixture of hydrophilic and hydrophobic resins in the presence of water would serve to compatibilize the network during photopolymerization. To test the hypothesis, we photopolymerized a 1 millimeter thick bar (1.5 g of 30:70 mole ratio of TEGDMA and EHA monomers), using 320-500 nanometer light at 45 milliwatts/centimeter², and photoinitiator DMPA at 1 weight percent in the presence of 150 microliters of water. The sample was observed to disintegrate during photopolymerization. See FIG. 6, Sample 2. A photocured sample in the absence of water was used as a control. See FIG. 6, Sample 1. However, the same formulation, when cured in the presence of water and 2.5 weight percent of the functionalized crosslinked copolymer poly(SA-co-DMAA5050) yields a crosslinked bar (FIG. 6, Sample 4) that has a flexural modulus (13±2 MPa) and flexural strength (1.3±0.3 MPa) that are comparable to those of the control sample (FIG. 6, Sample 1; flexural modulus=15±1 MPa; flexural strength=1.5±0.3). These results demonstrate the compatibilizing ability of the functionalized crosslinked copolymer in the presence of water.

As photopolymerization induced crosslinking limits the space for phase separation, the frustrated copolymers will self-assemble into predictable morphologies that will enhance the strength and toughness of the adhesive by forming a network of interpenetrated, chemically and physically cross-linked network components. This is illustrated by FIG. 7, which is a bar graph of flexural strength (in megapascals) as a function of sample type, where the samples are, from left to right, a control with no functionalized copolymer, a sample with 1 weight percent of a functionalized crosslinked random copolymer of stearyl acrylate and 2-ethylhexyl acrylate in a 25:75 mole ratio, and a sample with 1 weight percent of an unfunctionalized uncrosslinked diblock copolymer of stearyl acrylate and 2-ethylhexyl acrylate in a 1:1 mole ratio. To generate the flexural strength results shown in FIG. 7, a control formulation containing MMA/PMMA/TEGDMA (56:14:30 by weight percent, thickness=2 millimeters) and the photoinitiator DMPA at 1 weight percent was photocrosslinked via exposure to 320-500 nanometer light at 45 milliwatts/centimeter² for 180 seconds, yielding>75% double-bond conversion. Flexural modulus values were determined according to ASTM D790-17 using a test sample with dimensions 2 millimeter thickness by 2 millimeter width by 25 millimeters length, a 5 kilonewton load cell, a cross-span of 20 millimeters where the actuator is in the center of the span and extends downward at a rate of 1 millimeter/minute, the break threshold is 1 newton, and the break sensitivity is 60%. The photocrosslinked control sample exhibited a flexural strength of 44±5 megapascals. Addition of 1 weight percent of Random (middle bar in FIG. 7) or Block (right bar in FIG. 7) stearyl acrylate/2-ethylhexyl acrylate copolymer to the polymerizable formulation resulted in a greater than 50% increase in flexural strength (73±3 megapascals for random poly(SA-co-EHA2575), and 63±3 megapascals for diblock stearyl acrylate/2-ethylhexyl acrylate copolymer with SA units and EHA units in a 1:1 molar ratio.

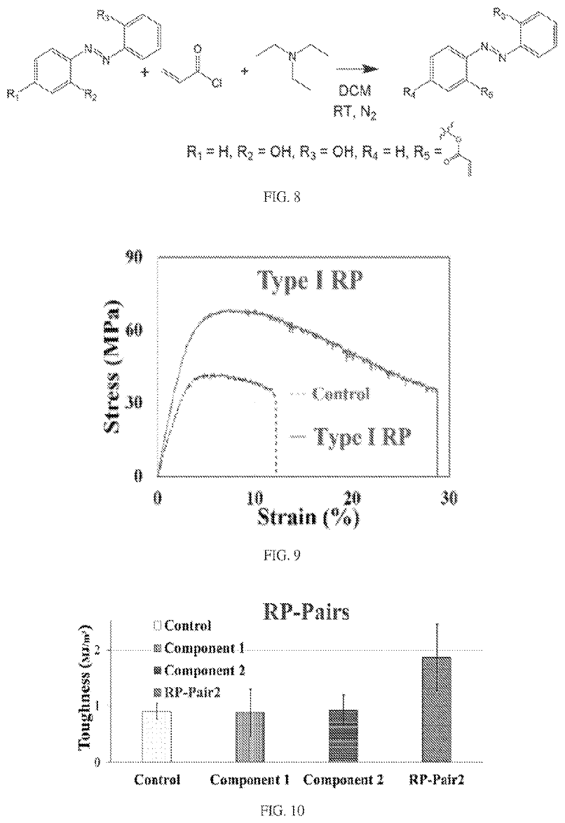

FIG. 9 is a stress versus strain curve for a Type I Reactive Polymer (Type I RP) and a control polymer. Flexural strength testing was conducted at 25° C. on an MTS Mini Bionix™ II hydraulic universal testing machine. Samples having dimensions 2 by 2 by 25 millimeters were placed in flexural clamps and tested at a strain rate of 1 millimeter/minute until failure. The control polymer was prepared from MMA, PMMA, and TEGDMA in a weight ratio of 56:14:30, respectively. The Type I Reactive Polymer was prepared by polymerizing 97.5 weight percent of MMA, PMMA, and TEGDMA in a weight ratio of 56:14:30, respectively, and 2.5 weight percent of the diblock copolymer designated Poly(SA-b-EHA) in Table 2. FIG. 9 demonstrates that, relative to the control polymer, the Type I Reactive Polymer exhibits a flexural strength at yield that is about 1.5 times greater, and a toughness that is about 3.5 times greater.

FIG. 10 illustrates the wet flexural strength advantage of a polymer composition (“RP-Pair2”) incorporating 5 weight percent of each of two different reactive polymers designated Component 1 and Component 2, compared to the same polymer composition containing 10 weight percent of either of the two different reactive polymers (“Component 1” and “Component 2”), or to the same polymer composition containing neither reactive polymer (“Control”). The base polymer composition was prepared from tetraethylene glycol dimethacrylate. The first reactive polymer was a copolymer of stearyl methacrylate, HEMA10 (having the structure below wherein n is 10)

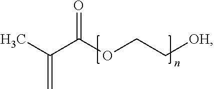

and tetraethylene glycol dimethacrylate in a weight ratio of 25:50:25, respectively, polymerized to about 65 percent conversion of double bonds. The second reactive polymer was a copolymer of stearyl methacrylate, mono-2-(methacryloyloxy)ethyl succinate, and tetraethylene glycol dimethacrylate in a weight ratio of 25:50:25, respectively, polymerized to about 65 percent conversion of double bonds. In FIG. 10, toughness values were determined after samples were maintained for 72 hours at 37° C. in deionized (Milli-Q) water. The results show that the RP-Pair2 polymer composition incorporating 5 weight percent of each of two different reactive polymers designated Component 1 and Component 2 exhibited a toughness approximately two times greater than each of the other polymer compositions.

FIG. 11 is a bar chart of cell viability (in units of percent) as a function of eluate concentration and associated cured composition. On curing, the reactive polymers are covalently bonded within dental resins. Based on the monomers and high double bond conversions achieved on curing, the cytocompatibility of the parent dental networks should not be affected by the addition of reactive polymers. To establish cytocompatibility, ISO 10993-5 protocols for adhesives were followed for both elution tests and direct contact tests using L929 mouse fibroblast cells. The “Control” sample was the polymerization product of TEGDMA and HEMA in a 60:40 weight ratio. The “Type I” sample was the polymerization product of 10 weight percent of a random copolymer of stearyl acrylate and a high molecular weight aliphatic urethane diacrylate obtained as EBECRYL™ 230 from AllNex in a 75:25 weight ratio and polymerized to 70 percent double bond conversion, and 90 weight percent TEGDMA and HEMA in a 60:40 weight ratio. The “Type II” sample was the polymerization product of 10 weight percent of a random copolymer of stearyl acrylate, HEMA5 (having the structure below wherein n is 5)

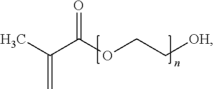

and TEGDMA in a 50:25:25 weight ratio and polymerized to 70 percent double bond conversion, and 90 weight percent TEGDMA and HEMA in a 60:40 weight ratio. Following 1, 3, and 7 days of incubation of the cured reactive polymer+resin (TEGDMA/HEMA, 60/40) discs (diameter=6.5 millimeters, thickness=0.8 millimeter) in extract medium (89 weight percent Dulbecco's Modified Eagle Medium (DMEM), 10 weight percent fetal bovine serum, 1 weight percent penicillin-streptomycin), the extract medium was subjected to the MTT assay of ISO 10993-5. The extract medium was diluted (80%, 40%, 20%) and added to L929 mouse fibroblast cells (ECACC 85011425) and incubated for an additional 24 hours after which the MTT reagent was added to the wells. The percent viability was calculated using the formula: % Cell Viability=(A_{Experimental group}/A_{control group})*100. For the direct contact tests, L929 cells were incubated for 72 hours with the substrates before the MTT reagent was added. All samples tested were classified as cytocompatible, showing greater than 80% cell viability in both tests.

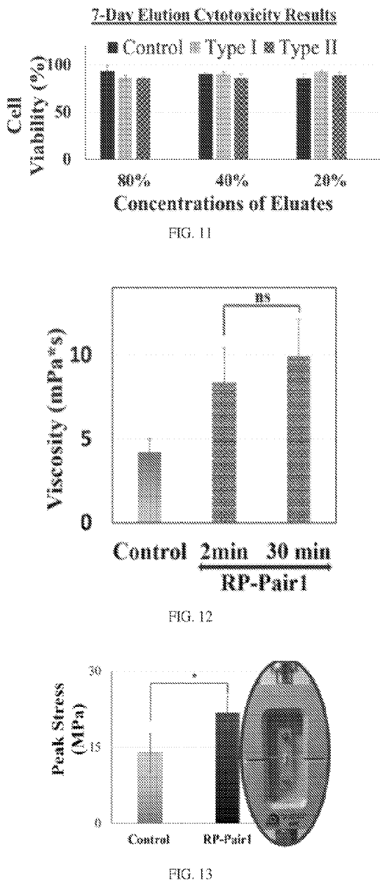

FIG. 12 is a bar chart of viscosity (in units of millipascal-seconds) as a function of composition. This figure illustrates that the pairs of reactive polymers do not form secondary bonds or interact with each other in a way that increases the viscosity of the polymerizable composition prior to polymerization. The “Control” composition was tetraethylene glycol dimethacrylate (TEGDMA). The mixture designated “RP-Pair1” consisted of 5 weight percent of a reactive copolymer produced by polymerization of stearyl methacrylate, HEMA5, and tetraethylene glycol dimethacrylate in 50:25:25 mole ratio to 65% conversion of double bonds; 5 weight percent of a reactive copolymer produced by polymerization of stearyl methacrylate, methacryloyloxyethyl succinate (MES; having the structure shown below)

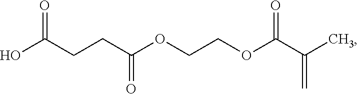 [SMILES: C=C(C)C(=O)OCCOC(=O)CCC(=O)O]

and tetraethylene glycol dimethacrylate in 50:25:25 mole ratio to 65% conversion of double bonds; and 90 weight percent of TEGDMA. To study viscosity effects, 5 weight percent of each reactive polymer component of RP-Pair1 was kept separately within TEGDMA until just before mixing to avoid the possibility of secondary interactions that can increase resin viscosity prior to polymerization. It was observed that mixing the components doubled the viscosity of the monomer composition within two minutes, but the viscosity remained statistically unchanged after 30 minutes, indicating no further secondary-interaction-driven rise in viscosity.

FIG. 13 is a bar chart of peak stress (microtensile bond strength; in units of megapascals) as a function of composition after exposure to a sucrose-rich broth for 72 hours. The inset is a photographic image of the experimental apparatus used to determine microtensile bond strength. Combinations of reactive polymers (RP-Pairs) are designed to crosslink collagen tubules within the hybrid layer to enhance the strength of the dentin-adhesive interface. And while RP-Pairs are not antibacterial prepolymers, any material within the dentin-adhesive interface will be exposed to the ubiquitous presence of bacteria in the oral cavity. Therefore, microtensile bond strength (μTBS) in the presence of bacteria was studied. Streptococcus mutans (S. mutans) is known to degrade resin bonding in the hybrid layer. For our initial tests, human molars were etched (37% phosphoric acid, Scotchbond™ Etchant, 3M ESPE) for 15 seconds, and the adhesive layer, which consisted of 10 parts by weight RP-Pair1 in 90 parts by weight of a 60:40 weight/weight mixture of TEGDMA and HEMA, which was dispersed in a mixture of 10 parts by weight ethanol and 1 part by weight camphorquinone/ethyl 4-N,N-dimethylaminobenzoate (CQ/A), was polymerized (Elipar™ at 700 mW/cm², irradiated twice for 20 seconds each), and the composite Filtek™ Z-100 (3M ESPE) was placed and cured (2 millimeters in height, 2 layers), achieving ≥70% double-bond conversion in under 40 seconds of incremental light exposure. After 24 hours wet storage at 37° C. in Milli-Q water, samples were sliced using a water-cooled diamond saw (Isomet™, Buehler) into 2 by 2 millimeter bars and exposed to S. mutans colonies in Brain Heart Infusion broth media (BHI) at 10⁷ colony forming units (cfu) for three days. The tooth samples in BHI were exposed to fresh sucrose (1 weight percent) on days 1 and 3, while the media was changed every day. Subsequent μTBS tests of the samples revealed that the polymer with RP-Pair 1 had superior bond strength (20 megapascals) in comparison to the control at 13 millipascals (FIG. 13), indicating a strengthened dentin-adhesive interface.

These working examples demonstrate that functionalized copolymers containing long chain aliphatic groups can efficiently direct self-assemblies and deliver predictable self-strengthening morphologies within crosslinked substrates.

1. A functionalized crosslinked acrylate copolymer comprising:
15 to 63 mole percent of first units derived from a first monomer selected from the group consisting of C₁₀-C₂₄-alkyl (meth)acrylates, C₁₀-C₂₄-alkenyl (meth)acrylates, C₁₀-C₂₄-alkyl (meth)acrylamides, C₁₀-C₂₄-alkenyl (meth)acrylamides, and combinations thereof;
35 to 83 mole percent of second units derived from a second monomer selected from the group consisting of (meth)acrylic acid, C₁-C₈-alkyl (meth)acrylates, C₁-C₈-alkyl (meth)acrylamides, 2-carboxyethyl (meth)acrylate, C₂-C₈-hydroxyalkyl (meth)acrylates, C₂-C₈-hydroxyalkyl (meth)acrylamides, ethylene glycol methyl ether (meth)acrylate, ethylene glycol methyl ether (meth)acrylamide, mono-2-(methacryloyloxy)ethyl maleate, mono-2-(methacryloyloxy)ethyl succinate, itaconic acid, 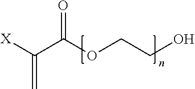
wherein X is hydrogen or methyl, and n is 2, 3, 4, 5, 6, 7, 8, 9, or 10, and combinations thereof; and
2 to 50 mole percent of third units derived from a third monomer selected from the group consisting of C₁-C₆-alkylene bis((meth)acrylate)s, C₁-C₆-alkylene bis((meth)acrylamide)s, ethylene glycol di(meth)acrylate, diethylene glycol di(meth)acrylate, triethylene glycol di(meth)acrylate, tetraethylene glycol di(meth)acrylate, bisphenol A glycerolate di(meth)acrylate, diurethane di(meth)acrylate, trimethylolpropane tri(meth)acrylate, di(trimethylolpropane) tetra(meth)acrylate, polycaprolactone di(meth)acrylate, polycaprolactone tri(meth)acrylate, and combinations thereof;
wherein the functionalized crosslinked acrylate copolymer comprises 0.6 to 25 mole percent of reactive functional groups selected from the group consisting of (meth)acrylate, (meth)acrylamido, and combinations thereof; and
wherein all mole percent values are based on total moles of first units, second units, and third units in the functionalized crosslinked acrylate copolymer.

2. The functionalized crosslinked acrylate copolymer of claim 1, wherein the first monomer is selected from the group consisting of C₁₂-C₂₂-alkyl (meth)acrylates, and combinations thereof.

3. The functionalized crosslinked acrylate copolymer of claim 1, wherein the second monomer is selected from the group consisting of C₁-C₈-alkyl (meth)acrylates, C₁-C₈-alkyl (meth)acrylamides, 2-carboxyethyl (meth)acrylate, C₂-C₈-hydroxyalkyl (meth)acrylates, C₂-C₈-hydroxyalkyl (meth)acrylamides, ethylene glycol methyl ether (meth)acrylate, ethylene glycol methyl ether (meth)acrylamide, mono-2-(methacryloyloxy)ethyl maleate, mono-2-(methacryloyloxy)ethyl succinate, itaconic acid, 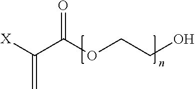
wherein X is hydrogen or methyl, and n is 2, 3, 4, 5, 6, 7, 8, 9, or 10, and combinations thereof.

4. The functionalized crosslinked acrylate copolymer of claim 1, wherein the second monomer is selected from the group consisting of C₁-C₈-alkyl (meth)acrylates, C₁-C₈-alkyl (meth)acrylamides, 2-carboxyethyl (meth)acrylate, C₂-C₈-hydroxyalkyl (meth)acrylates, C₂-C₈-hydroxyalkyl (meth)acrylamides, ethylene glycol methyl ether (meth)acrylate, ethylene glycol methyl ether (meth)acrylamide, mono-2-(methacryloyloxy)ethyl maleate, mono-2-(methacryloyloxy)ethyl succinate, itaconic acid, 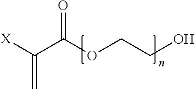
wherein X is hydrogen or methyl, and n is 2, 3, 4, 5, 6, 7, 8, 9, or 10, and combinations thereof, provided that if the second monomer comprises a C₁-C₈-alkyl (meth)acrylamide, it excludes N,N-dimethylacrylamide.

5. The functionalized crosslinked acrylate copolymer of claim 1, wherein the second monomer is selected from the group consisting of acrylic acid, ethyl acrylate, butyl acrylate, 2-ethylhexyl acrylate, 2-carboxyethyl acrylate, ethylene glycol methyl ether acrylate, N,N-dimethyl acrylamide, N-iso-propyl acrylamide, N-tert-butyl acrylamide, N-hydroxyethyl acrylamide, 2-hydroxypropyl methacrylamide, N-[tris(hydroxymethyl)methyl]acrylamide, 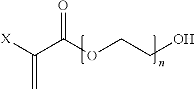
wherein X is hydrogen or methyl, and n is 2, 3, 4, 5, 6, 7, 8, 9, or 10, and combinations thereof.

6. The functionalized crosslinked acrylate copolymer of claim 1, wherein the third monomer is selected from the group consisting of N,N′-methylenebis((meth)acrylamide), 1,4-butanediol di(meth)acrylate, 1,6-hexanediol di(meth)acrylate, ethylene glycol di(meth)acrylate, diethylene glycol di(meth)acrylate, triethylene glycol di(meth)acrylate, and combinations thereof.

7. The functionalized crosslinked acrylate copolymer of claim 1, wherein the functionalized crosslinked acrylate copolymer is a functionalized crosslinked acrylate random copolymer comprising randomly dispersed first units and second units.

8. The functionalized crosslinked acrylate copolymer of claim 1, further comprising 0.5 to 5 mole percent of hydroxy-functionalized azobenzene groups.

9. The functionalized crosslinked acrylate copolymer of claim 1, comprising:
15 to 60 mole percent of the first units; wherein the first monomer is selected from the group consisting of C₁₂-C₂₂-alkyl (meth)acrylates, and combinations of C₁₂-C₂₂-alkyl (meth)acrylates;
15 to 60 mole percent of the second units; wherein the second monomer is selected from the group consisting of acrylic acid, ethyl acrylate, butyl acrylate, 2-ethylhexyl acrylate, 2-carboxyethyl acrylate, ethylene glycol methyl ether acrylate, N,N-dimethyl acrylamide, N-iso-propyl acrylamide, N-tert-butyl acrylamide, N-hydroxyethyl acrylamide, 2-hydroxypropyl methacrylamide, N-[tris(hydroxymethyl)methyl]acrylamide, and combinations thereof; and
15 to 35 mole percent of the third units; wherein the third monomer is selected from the group consisting of N,N′-methylenebis((meth)acrylamide), ethylene glycol di(meth)acrylate, 1,4-butanediol di(meth)acrylate, 1,6-hexanediol di(meth)acrylate, diethylene glycol di(meth)acrylate, triethylene glycol di(meth)acrylate, and combinations thereof;
wherein the functionalized crosslinked acrylate copolymer comprises 2 to 20 mole percent of the reactive functional groups.

10. The functionalized crosslinked acrylate copolymer of claim 1, comprising:
15 to 60 mole percent of the first units; wherein the first monomer is selected from the group consisting of C₁₂-C₂₂-alkyl (meth)acrylates, and combinations of C₁₂-C₂₂-alkyl (meth)acrylates;
15 to 60 mole percent of the second units; wherein the second monomer is selected from the group consisting of acrylic acid, 2-carboxyethyl acrylate, N,N-dimethyl acrylamide, N-iso-propyl acrylamide, N-tert-butyl acrylamide, N-hydroxyethyl acrylamide, 2-hydroxypropyl methacrylamide, N-[tris(hydroxymethyl)methyl]acrylamide, and combinations thereof; and
15 to 35 mole percent of the third units; wherein the third monomer is selected from the group consisting of N,N′-methylenebis((meth)acrylamide), ethylene glycol di(meth)acrylate, 1,4-butanediol di(meth)acrylate, 1,6-hexanediol di(meth)acrylate, diethylene glycol di(meth)acrylate, triethylene glycol di(meth)acrylate, and combinations thereof;
wherein the functionalized crosslinked acrylate copolymer comprises 2 to 20 mole percent of the reactive functional groups.

11. A method of forming a functionalized crosslinked acrylate copolymer, comprising:
partially copolymerizing a monomer mixture comprising
a first monomer selected from the group consisting of C₁₀-C₂₄-alkyl (meth)acrylates, C₁₀-C₂₄-alkenyl (meth)acrylates, C₁₀-C₂₄-alkyl (meth)acrylamides, C₁₀-C₂₄-alkenyl (meth)acrylamides, and combinations thereof;
a second monomer selected from the group consisting of (meth)acrylic acid, C₁-C₈-alkyl (meth)acrylates, C₁-C₈-alkyl (meth)acrylamides, 2-carboxyethyl (meth)acrylate, C₂-C₈-hydroxyalkyl (meth)acrylates, C₂-C₈-hydroxyalkyl (meth)acrylamides, ethylene glycol methyl ether (meth)acrylate, ethylene glycol methyl ether (meth)acrylamide, mono-2-(methacryloyloxy)ethyl maleate, mono-2-(methacryloyloxy)ethyl succinate, itaconic acid, 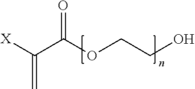
wherein X is hydrogen or methyl, and n is 2, 3, 4, 5, 6, 7, 8, 9, or 10, and combinations thereof; and
a third monomer selected from the group consisting of C₁-C₆-alkylene bis((meth)acrylate)s, C₁-C₆-alkylene bis((meth)acrylamide)s, diethylene glycol di(meth)acrylate, triethylene glycol di(meth)acrylate, tetraethylene glycol di(meth)acrylate, bisphenol A diglycidyl ether di(meth)acrylate, diurethane di(meth)acrylate, trimethylolpropane tri(meth)acrylate, di(trimethylolpropane) tetra(meth)acrylate, polycaprolactone di(meth)acrylate, polycaprolactone tri(meth)acrylate, and combinations thereof
to form a functionalized crosslinked acrylate copolymer comprising
15 to 63 mole percent of first units derived from the first monomer,
35 to 83 mole percent of second units derived from the second monomer, and
2 to 50 mole percent of third units derived from the third monomer;
wherein of the 2 to 50 mole percent of third units derived from the third monomer, 0.6 to 25 mole percent have at least one pendant (meth)acryloyloxy or (meth)acrylamido group, and 25 to 49.4 mole percent have zero pendant (meth)acryloyloxy or (meth)acrylamido groups; and
wherein all mole percent values are based on total moles of first units, second units, and third units in the functionalized crosslinked acrylate copolymer.

12. The method of claim 11, wherein the partially copolymerizing comprises polymerizing 50 to 80 mole percent of polymerizable groups contributed by the first monomer, second monomer, and third monomer.

13. The method of claim 11,
wherein the second monomer comprises the C₂-C₈-hydroxyalkyl (meth)acrylate, the C₂-C₈-hydroxyalkyl (meth)acrylamide, or a combination thereof, and at least a portion of the second units comprise pendant hydroxyl groups; and
wherein the method further comprises reacting at least a portion of the pendant hydroxyl groups with a reagent capable of producing a pendant (meth)acrylate group.

14. The method of claim 11, wherein the functionalized crosslinked acrylate copolymer comprises:
20 to 50 mole percent of the first units; wherein the first monomer is selected from the group consisting of C₁₂-C₂₂-alkyl (meth)acrylates, and combinations of C₁₂-C₂₂-alkyl (meth)acrylates;
45 to 75 mole percent of the second units; wherein the second monomer is selected from the group consisting of acrylic acid, ethyl acrylate, butyl acrylate, 2-ethylhexyl acrylate, 2-carboxyethyl acrylate, ethylene glycol methyl ether acrylate, N,N-dimethyl acrylamide, N-iso-propyl acrylamide, N-tert-butyl acrylamide, N-hydroxyethyl acrylamide, 2-hydroxypropyl methacrylamide, N-[tris(hydroxymethyl)methyl]acrylamide, 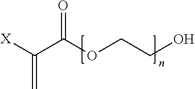
wherein X is hydrogen or methyl, and n is 2, 3, 4, 5, 6, 7, 8, 9, or 10, and combinations thereof; and
5 to 35 mole percent of the third units; wherein the third monomer is selected from the group consisting of N,N′-methylenebis((meth)acrylamide), ethylene glycol di(meth)acrylate, 1,4-butanediol di(meth)acrylate, 1,6-hexanediol di(meth)acrylate, diethylene glycol di(meth)acrylate, triethylene glycol di(meth)acrylate, and combinations thereof;
wherein the functionalized crosslinked acrylate copolymer comprises 2 to 20 mole percent of the reactive functional groups.

15. A curable composition comprising:
the functionalized crosslinked acrylate copolymer of claim 1; and
a copolymerizable monomer.

16. The curable composition of claim 15, comprising, based on the total weight of the curable composition,
2 to 25 weight percent of the functionalized crosslinked acrylate copolymer; and
75 to 98 weight percent of the copolymerizable monomer; wherein the copolymerizable monomer comprises, based on 100 parts by weight of the copolymerizable monomer,
80 to 95 parts by weight of a fourth monomer selected from the group consisting of (meth)acrylic acid, C₁-C₈-alkyl (meth)acrylates, C₁-C₈-alkyl (meth)acrylamides, 2-carboxyethyl (meth)acrylate, C₂-C₈-hydroxyalkyl (meth)acrylates, C₂-C₈-hydroxyalkyl (meth)acrylamides, ethylene glycol methyl ether (meth)acrylate, ethylene glycol methyl ether (meth)acrylamide, mono-2-(methacryloyloxy)ethyl maleate, mono-2-(methacryloyloxy)ethyl succinate, itaconic acid, and combinations thereof; and
5 to 20 parts by weight of a fifth monomer selected from the group consisting of diethyleneglycol di(meth)acrylate, triethyleneglycol di(meth)acrylate, tetraethyleneglycol di(meth)acrylate, bisphenol A glycerolate di(meth)acrylate, diurethane di(meth)acrylate, C₁-C₆-alkylene bis((meth)acrylate)s, C₁-C₆-alkylene bis((meth)acrylamide)s, trimethylolpropane tri(meth)acrylate, di(trimethylolpropane) tetra(meth)acrylate, polycaprolactone di(meth)acrylate, polycaprolactone tri(meth)acrylate, and combinations thereof.

17. The curable composition of claim 15, wherein the functionalized crosslinked acrylate copolymer comprises:
15 to 60 mole percent of the first units; wherein the first monomer is selected from the group consisting of C₁₂-C₂₂-alkyl (meth)acrylates, and combinations of C₁₂-C₂₂-alkyl (meth)acrylates;
15 to 60 mole percent of the second units; wherein the second monomer is selected from the group consisting of acrylic acid, ethyl acrylate, butyl acrylate, 2-ethylhexyl acrylate, 2-carboxyethyl acrylate, ethylene glycol methyl ether acrylate, N,N-dimethyl acrylamide, N-iso-propyl acrylamide, N-tert-butyl acrylamide, N-hydroxyethyl acrylamide, 2-hydroxypropyl methacrylamide, N-[tris(hydroxymethyl)methyl]acrylamide, 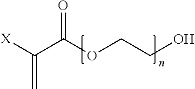
wherein X is hydrogen or methyl, and n is 2, 3, 4, 5, 6, 7, 8, 9, or 10, and combinations thereof;
15 to 35 mole percent of the third units; wherein the third monomer is selected from the group consisting of N,N′-methylenebis((meth)acrylamide), ethylene glycol di(meth)acrylate, 1,4-butanediol di(meth)acrylate, 1,6-hexanediol di(meth)acrylate, diethylene glycol di(meth)acrylate, triethylene glycol di(meth)acrylate, and combinations thereof;
wherein the functionalized crosslinked acrylate copolymer comprises 2 to 20 mole percent of the reactive functional groups.

18. The curable composition of claim 15,
wherein the functionalized crosslinked acrylate copolymer comprises
a first functionalized crosslinked acrylate copolymer comprising second units derived from a second monomer selected from the group consisting of C₂-C₈-hydroxyalkyl (meth)acrylates, C₂-C₈-hydroxyalkyl (meth)acrylamides, 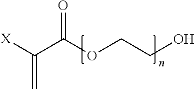
wherein X is hydrogen or methyl, and n is 2, 3, 4, 5, 6, 7, 8, 9, or 10, and combinations thereof; and
a second functionalized crosslinked acrylate copolymer comprising second units derived from a second monomer selected from the group consisting of (meth)acrylic acid, mono-2-(methacryloyloxy)ethyl maleate, mono-2-(methacryloyloxy)ethyl succinate, itaconic acid, and combinations thereof;
wherein the curable composition comprises, based on the total weight of the curable composition, 2 to 15 weight percent of the first functionalized crosslinked acrylate copolymer and 2 to 15 weight percent of the second functionalized crosslinked acrylate copolymer.

19. A cured composition comprising the product of curing the curable composition of claim 15.

20. A curable composition comprising:
an unfunctionalized uncrosslinked acrylate block copolymer comprising a first block and a second block; wherein
the first block comprises first units derived from a first monomer selected from the group consisting of C₁₀-C₂₄-alkyl (meth)acrylates, C₁₀-C₂₄-alkenyl (meth)acrylates, C₁₀-C₂₄-alkyl (meth)acrylamides, C₁₀-C₂₄-alkenyl (meth)acrylamides, and combinations thereof; and
the second block comprises second units is derived from a second monomer selected from the group consisting of (meth)acrylic acid, C₁-C₈-alkyl (meth)acrylates, C₁-C₈-alkyl (meth)acrylamides, 2-carboxyethyl (meth)acrylate, C₂-C₈-hydroxyalkyl (meth)acrylates, C₂-C₈-hydroxyalkyl (meth)acrylamides, ethylene glycol methyl ether (meth)acrylate, ethylene glycol methyl ether (meth)acrylamide, mono-2-(methacryloyloxy)ethyl maleate, mono-2-(methacryloyloxy)ethyl succinate, itaconic acid, 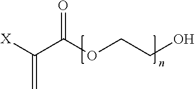
wherein X is hydrogen or methyl, and n is 2, 3, 4, 5, 6, 7, 8, 9, or 10, and combinations thereof;
wherein the unfunctionalized uncrosslinked acrylate block copolymer comprises 15 to 65 mole percent of first units and 35 to 85 mole percent of second units, based on the total mole percent of first units and second units in the unfunctionalized uncrosslinked acrylate block copolymer; and
a polymerizable monomer comprising a sixth monomer comprising two or more reactive groups selected from the group consisting of (meth)acryloyloxy groups, (meth)acrylamido groups, and combinations thereof.

21.-22. (canceled)
